(19) United States
(12) Patent Application Publication
(10) Pub. No.: US 2005/0239738 A1
(43) Pub. Date: Oct. 27, 2005

(54) Genetic inhibition of epidermal growth factor receptor function and carcinoma cell radiosensitization

(76) Inventors: Rupert Schmidt-Ullrich, Richmond, VA (US); C. Kristoffer Valerie, Midlothian, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 11/147,349

(22) Filed: Jun. 8, 2005

Related U.S. Application Data
(62) Division of application No. 09/714,790, filed on Nov. 17, 2000.

(51) Int. Cl. A61K 48/00

(52) U.S. Cl. 514/44

(57) ABSTRACT

The present invention is drawn to a method of suppressing the phenomenon of accelerated repopulation, i.e. the proliferation of cells upon exposure to a clinically relevant dose of radiation energy. More specifically, the invention provides a method of radiosensitizing cancer cells by administering an expressible nucleic acid molecule encoding a mutant form of epidermal growth factor receptor. In a preferred embodiment of the invention, the mutant form of epidermal growth factor receptor is EGFR-CD533, a C-terminal truncated epidermal growth factor receptor that lacks mitogenic and transformation activity. The method of this invention thus constitutes a gene therapy approach to the radiosensitization of cancer cells.

### CROSS-REFERENCE TO RELATED APPLICATIONS

This application claims the benefit of Provisional Application Serial No. 60/165,940, entitled “Disruption of EGFR Function through Expression of Dominant Negative EGFR-CD533 (or Other Inhibitors) as a Tool to Inhibit Carcinoma Cells' Proliferation in Response to Repeated Radiation Therapy Treatments and to Radiosensitize Such Cells,” filed on Nov. 17, 1999, which is herein incorporated by reference.

This invention was made in part using funds from grants from the United States Public Health Service (PHS) having grant number CA65986 and CA72955. The government may have certain rights in this invention.

### BACKGROUND OF THE INVENTION

1. Field of the Invention

The present invention relates to methods for radiosensitizing cancer cells. The methods encompass both the direct radiosensitization of cancer cells, and the inhibition of accelerated repopulation, the radiation-induced proliferation of cancer cells that occurs during radiation therapy. More particularly, the present invention relates to methods for radiosensitizing cancer cells by administering a dominant negative form of epidermal growth factor receptor to the cancer cells.

2. Background of the Invention

In the United States, cancer is the second leading cause of death behind cardiovascular disease, and it is projected that cancer will become the leading cause of death within a few years, in spite of many recent advancements in treatment of the disease. (Gibbs et al., 2000).

Radiation therapy is a mainstay of cancer treatment. When used in combination with surgery and/or chemotherapy, as is generally the case, remarkable success is often achieved in treating a wide variety of cancers. This is especially true if the cancer is a tumor at an early, localized stage. However, the effectiveness of radiation therapy can be drastically attenuated by a well-documented phenomenon known as “accelerated repopulation.”

Accelerated repopulation refers to the powerful compensatory proliferation response of tumor cells which is observed during fractionated (repeated) radiation exposures during clinically applied radiotherapy. In other words, rather than being killed by the radiation, some tumor cells (especially toward the end of a fractionated course of radiotherapy) actually begin to proliferate at an accelerated rate in response to radiation, completely nullifying the purpose of the treatment. This proliferation response has, not surprisingly, been demonstrated to adversely affect tumor control and patient survival.

It would be highly desirable to have available an effective method for sensitizing cancer cells to radiation in general, and in particular, for overcoming the phenomenon of accelerated repopulation. Such methods would render cancer cells more susceptible to killing by radiation, thereby improving tumor control and increasing patient survival rates.

### SUMMARY OF THE INVENTION

The present invention is drawn to a method of radiosensitizing cancer cells in general, and in particular to a method of suppressing accelerated repopulation, i.e. the proliferation of cancer cells upon exposure to a clinically relevant dose of radiation energy. More specifically, the invention provides a method of radiosensitizing cancer cells by administering to cancer cells an expressible nucleic acid molecule encoding a mutant form of the epidermal growth factor receptor (EGFR) in order to disrupt the receptor's function. Particularly, the method comprises delivering an expressible nucleic acid encoding EFGR-CD533, a C-terminal truncated epidermal growth factor receptor which lacks mitogenic and transformation activity.

Accordingly, the present invention satisfies the need to sensitize cancer cells for radiotherapy and provides related advantages as well. Still further objects and advantages of present invention will become apparent from a consideration of the drawings and ensuing description. It will be apparent to one skilled in the art that various substitutions and modifications may be made to the invention disclosed herein without departing from the scope and the merit of the invention.

### BRIEF DESCRIPTION OF THE DRAWINGS

FIGS. 1A and 1B. Doxycycline-inducibility of EGFR-CD533 in MCF-TR5-EGFR-CD533 and MDA-TR15-EGFR-CD533 cells. The mRNA was quantified for MCF-TR5-EGFR-CD533 cells by RNase protection assays (FIG. 1A) and by Northern analyses for MDA-TR15-EGFR-CD533 cells (FIG. 1B). The expression of the 110 kD EGFR-CD533 protein was determined by a ³⁵S-methionine, 50μ Ci/ml for 6 h, labeling 48 h after Dox exposure at 1 μg/ml followed by immunoprecipitation and size-fractionation by SDS-PAGE. The 170 kD EGFR-WT was identified in cell lysates from ³⁵S-methionine labeled A431 cells. The results shown are representative of 3 independent experiments.

FIG. 2A-C. Kinetics of Doxycycline-inducibility of EGFR-CD533 in MDA-TR15-EGFR-CD533 cells. For MDA-TR15-EGFR-CD533 cells, the Dox concentration dependency of EGFR-CD533 induction was established (FIG. 2A). The induction (FIG. 2B) and decay (FIG. 2C) kinetics of EGFR-CD533 expression after addition and removal of Dox were also monitored by ³⁵S-methionine labeling using 6 h pulses of the label at the designated times. Autoradiograms were scanned using Jandel Scientific (San Rafael, Calif.) software. The results shown are representative of 3 independent experiments.

FIG. 3A-C. Expression of EGFR-WT and erbB-2 as function of duration of EGFR-CD533 expression in MDA-TR15-EGFR-CD533 cells. FIG. 3A: Cell lysates were size fractionated by SDS-PAGE and immunoblotted with anti-EGFR Ab14. The lower panel is a longer exposure of the same blot at the EGFR protein band. FIG. 3B: EGFR-WT was immunoprecipitated from lysates of MDA-TR15-EGFR-CD533 cells (see Methods), size fractionated by SDS-PAGE and immunoblotted with E12120, a mAb specific for the activated form of EGFR. FIG. 3C: ErbB-2 was immunoprecipitated from cell lysates and the resulting blot was probed with anti-erbB-2.

FIGS. 4A and 4B. Radiation-induced activation of EGFR-WT. FIG. 4A: Cells lysates from MDA-TR15-EGFR-CD533 cells, at specified times after exposure of cells to 2 Gy or 2 ng/ml of EGF, were immunoprecipitated with anti-EGFR mAb and size fractionated by SDS-PAGE; Western blots were reacted with anti-phospho-Tyr mAb and the reaction quantified by chemiluminescence and densitometric scanning. The right eight lanes are from cells exposed to 1 μg/ml Dox for 48 h prior to irradiation or EGF treatment. The left eight lanes are from cells not treated with Dox. FIG. 4B: The same experiment as in 4A but with MDA-TR15-Luc27 cells.

FIG. 5A-C. MAPK activity in MDA-TR15-/MCF-TR5-EGFR-CD533 cells after radiation exposure as a function of EGFR-CD533 expression. FIG. 5A: The MAPK activity profile for MDA-TR15-EGFR-CD533 cells, expressed relative to pretreatment controls (1.0) after treatment with 2 Gy of radiation. Cells exposed to Dox, 1 μg/ml for 48 h (▪) are compared to cells not treated with Dox (●). FIG. 5B: MDA-TR15-Luc27 cells with the p-UHD-13-3 luciferase control vector (see Methods) treated with radiation as in FIG. 5A. FIG. 5C: MCF-TR5-EGFR-CD533 cells treated as described in 5A. The data from this single experiment, run in triplicate, are representative of 4 independent experiments.

FIGS. 6A and 6B. Effects of EGFR-CD533 expression during and after irradiation on relative growth of MDA-TR15-EGFR-CD533 cells. Cells with or without exposure to Dox at 1 μg/ml for 24 h were irradiated with 2 Gy for 5 consecutive days followed by growth analyses on days 1, 4, and 7 after irradiation. The treatment scheme is depicted in the top portion of the figure. The relative growth, as assessed by MTT assay (see Methods), was quantified as a function of EGFR-CD533 expression by Dox under control conditions (FIG. 6A) and in combination with irradiation (FIG. 6B). The growth retardation mediated by EGFR-CD533 expression is significant on days 4 and 7 with p-values of 0.01 and 0.005 (FIG. 6A), respectively; this growth inhibition is dramatically amplified in the irradiation experiments with p-values for days 4 and 7 of <0.002 and <0.0001, respectively. Each data point represents the average±S.E.M. of eight samples. The results from a single experiment are representative of 5 independent experiments from which the statistical data have been derived. The cell growth is expressed as absorbance relative to that on day 1 of the growth assay.

FIG. 7A-D. Effects of dominant negative EGFR CD533 expression on MAPK and JNK1 activation after it single radiation exposure of 2 Gy. MDA-TR15-EGFR-CD533 cells, FIGS. 7A and 7C, were treated with (●) or without (▪) Dox for 48 h prior to irradiation (2 Gy) for the induction of EGFR-CD533 expression. The parental control cell line MDA-TR15, FIGS. 7B and 7D, lacking a Dox inducible gene was also treated with (●) or without (▪) Dox for 48 h prior to irradiation (2 Gy). At the indicated times after irradiation, MAPK and JNK1 activities were measured and expressed relative to basal activities prior to irradiation. For experimental details see Materials and Methods. (FIG. 7A) MAPK: (FIG. 7C) JNK1. The actual radioactive Counts were converted into fold activation. MAPK activity at time=0 were 12850±800 c.p.m. and 13104±700 c.p.m. for cells without and with expression EGFR-CD533, respectively. The corresponding JNK1 activities at time=0 were 5300±85O c.p.m. and 4900±650 c.p.m. under control conditions or conditions of EGFR-CD533 expression respectively. (FIG. 7B) MAPK: (FIG. 7D) JNK1. The actual radioactive counts converted to fold activation. MAPK activity at time 1 was 14 000±810 c.p.m. for control MDA-TR15 cells and 16000±1600 c.p.m. for Dox treated MD-TR15 cells: the corresponding JNK1 activities at time=0 was 4750±400 c.p.m. and 3500±300 c.p.m. respectively. The results shown give average values from three independent experiments.

FIG. 8A-C. Effects of EGFR-CD533 expression and MEK1/2 inhibition with PD98059 on JNK1 activity after irradiation. MDA-TR15-EGFR-CD533 cells were treated with or without Dox for 48 hr or with or without PD98059 for 2 hr prior to irradiation (21 Gy) for EGFR-CD533 expression and MAPK1/2 inhibition, respectively. At indicated times after irradiation, JNK1 and MAPK enzymatic activity was measured and expressed relative to basal activity prior to irradiation. (For details see Materials and Methods in Example 2). FIG. 8A: MAPK, (▪−PD98059. ●+PD98059); FIG. 8B: JNK1, (▪−PD98059, ●+PD98059); FIG. 8C: JNK1, (▪−PD98059, −EGFR-CD533,●+PD98059,+EGFR-CD533). The actual radioactive counts were converted to fold activation. MAPK activity at time=0 was 15450±775 c.p.m. for cells without PD98059, and was 16900±575 c.p.m. for cells with PD98059, JNK1 activity at time=0 was 3350±250 c.p.m. for cells without PD98059, and was 3100±200 c.p.m. for cells with PD98059. JNK1 activity at time=0 was 3350±250 c.p.m. for cells without EGFR-CD533 expression and MEK1/2 inhibition and was 3475±300 c.p.m, for cells expressing EGFR-CD533 with simultaneous MEK1/2 inhibition. The results shown give average values from three separate experiments.

FIG. 9A-C. Effects of Ras inhibition by FTI on radiation-induced MAPK and JNK1 activation. MDA-TR15-EGFR-CD533 cells were treated with (●) or without (▪) FTI at 50 μM for 24 h prior to irradiation with 2 Gy and the MAPK and JNK1 activities assessed (FIGS. 9A and 9B). At the indicated times after irradiation. MAPK and JNK1 enzymatic activities were measured and expressed relative to basal levels prior to FTI treatment. Effects of MEK1/2 inhibition on basal JNK1 activity was assessed upon treatment with (●) or without (●) FTI 24 h prior to treatment of cells with 10 μM PD98059 in the absence of radiation exposure (9C). For details see Materials and Methods of Example 2. FIG. 9A: MAPK; FIG. 9B: JNK1. The actual radioactive counts converted to fold activation. MAPK activity at time=0 was 14400±875 c.p.m. for control conditions and 11675±175 c.p.m. for cells treated with FTI; the corresponding values for JNK1 activities at time=0 were 3475±275 c.p.m. and 3975±150 c.p.m. respectively. FIG. 9C: At the indicated times immediately after addition of PD9809 the JNK1 activity was determined. The actual radioactive counts converted to fold activation, for JNK1 activity at time=0 were 3475±275 c.p.m. for control conditions and 3975±150 c.p.m, for FTI treated cells. The results shown are an average of three independent experiments.

FIG. 10A-B. Inhibition of MAPK signaling and c-Jun Ser 63 phosphorylation in vivo. MDA-TR15-EGFR-CD533 cells were treated without (control, left panels) or with (PD98059, right panels) 10 μM PD98059 for 2 h and were irradiated with 2 Gy thereafter. Protein and Ser 63 phosphorylation levels of c-Jun were determined up to 60 min after irradiation. At the indicated time points in min. 100 μg total protein from each cell lysate were subjected to SDS-PAGE followed by Western blotting for c-Jun protein and Ser 63 phosphorylated c-Jun as described in Materials and Methods of Example 2. FIG. 10A: c-Jun protein. At the indicated time points after irradiation all cellular levels of c-Jun protein were expressed relative to the c-Jun protein level in un-irradiated cells, at time 0, under control conditions (a. left lane). Figure 10B: The alterations of Ser 63 phosphorylation of c-Jun were derived in a two-step process. First, the relative Ser 63 phosphorylation levels of c-Jun were expressed for all time points of both control conditions and PD98059 exposure relative to time 0 of the control (upper values in FIG. 10B); then the alterations of Ser 63 phosphorylation were quantified as the ratio of the respective relative phosphorylation over relative protein levels for each time point of control conditions and PD98059 exposure (lower values in FIG. 10B). The results are representative of two independent experiments

FIG. 11A-C. Effects of EGFR or MAPK function on radiation-induced apoptosis. Apoptosis rates of MDA-TR15-EGFR-CD533 cells with and without 2 Gy of radiation were determined after treatment with the MEKI/2 inhibitor PD98059 or expression of EGFR-CD533 or recombinant adenovirus N-terminal truncated c-Jun (Ad-TAM 67) expression. Apoptosis was assessed by TUNEL 24 h after irradiation as described in Materials and Methods of Example 2. FIG. 11A: Effects of radiation in conjunction with EGFR-CD533 expression or MEK1/2 on cellular apoptosis. FIG. 11B: Effects of radiation in conjunction with recombinant adenovirus control (null) or Ad-TAM 67 (TAM 67) expression on apoptosis. FIG. 11C: Effects of radiation on apoptosis in conjunction with TAM67 expression with and without inhibition of MEK1/2. *Conditions resulting in statistical enhancement above additivity at the P<0.05 levels. Data was pooled from three separate experiments and the proportion of apoptotic cells shown±s.e.m.

FIG. 12A-B. Effects of dominant negative EGFR-CD533 and MAPK inhibition on cell proliferation after repeated radiation exposures of 2 Gy. The repeated radiation exposure proliferation assay with MDA-TR15-EGFR-CD533 cells utilized the MTT assay for cell quantification: for details see Materials and Methods of Example 2. FIG. 12A: Cell proliferation with (●) or without (▪) expression of EGFR-CD533. FIG. 12B: Cell proliferation with (●) or without (▪) MEK1/2 inhibition. The assay included daily exposure of cells to 2 Gy of radiation over 5 consecutive days. Cell proliferation was monitored on days 1, 4, 7 after the last radiation exposure. Cell proliferation was quantified as incremental cell numbers relative to those on day 1. Errors represent±s.e.m. of six individual data points from a representative experiment representative for three independent experiments

FIG. 13. Transduction efficiency of Ad-EGFR-CD533 at increasing MOI. Transduction efficiencies of MDA cells with Ad-LacZ at MOI's between 5 and 100. Expression of β-gal was determined by x-gal staining 48 h after transduction. All additional experiments were performed at 50 MOI yielding an overall transduction rate >80% with <20% associated cytotoxicity.

FIG. 14. Kinetics of EGFR-CD533 expression. Ad-mediated expression of EGFR-CD533 as quantified by immunoblotting 24 and 48 h after transduction relative to wild-type EGFR expression. The EGFR-CD533 expression kinetics demonstrate detectable expression 24 h after transduction with Ad-EGFR-CD533 at 50 MOI, approaching maximum values by 48 h.

FIG. 15A-C. Radiation-induced EGFR Tyr-P at specified times after radiation exposure of 2 Gy in untransduced, control (Ad-LacZ) and Ad-EGFR-CD533 transduced MDA cells at 50 MOI. Western blots of immunoprecipitated EGFR with anti-phospho-Tyr mAb. The corresponding values of relative Tyr-P levels represent increases over the negative control. The results shown are representative of three independent experiments.

FIG. 16. Effects of EGFR-CD533 on radiation-induced MAPK activation. Time course of MAPK activity at specified times after radiation exposure of 2 Gy in MDA cells 48 h after transduction with Ad-LacZ or Ad-EGFR-CD533 at 50 MOI. The corresponding fold activation of MAPK activity represents changes over the negative control. The results shown are representative of two independent experiments.

FIGS. 17A and 17B. Radiation dose response analyses of MDA-EGFR-CD533 and MDA cells as a function of EGFR-CD533 over-expression. Clonogenic survival is determined after single radiation exposures between 1 and 8 Gy. The log survival was formed to the number of cells plated, after correcting for plating efficiency. The error bars represent the mean±S.E.M. in four independent experiments. 17A: Clonogenic survival of MDA-EGFR-CD533 cells under control conditions [−Dox=¤] or conditions of EGFR-CD533 expression [+Dox=⋄]. The significant radiosensitization under conditions of EGFR-CD533 expression is reflected in a D ratio of 1.5 (P<0.0001). 17B: Clonogenic survival of MDA cells under control conditions (mock infection) [¤]; or transduction with Ad-LacZ [Δ] or Ad-EGFR-CD533 [⋄] at 50 MOI. Relative to untreated controls there is no radiosensitization after transduction with Ad-LacZ; in contrast, transduction with Ad-EGFR-Cd533 resulted in significant radiosensitization as reflected in a D ratio of 1.4 (P<0.0001).

FIG. 18. Radiation responses of EGFR in vivo and expression of EGFR-CD533 in MDA tumor xenografts: 18A. Radiation-induced activation of EGFR after single doses of 4 Gy in MDA tumor xenografts 2 to 10 min after irradiation; immnunoblots of immunoprecipitated EGFR probed with an antiphospho-Tyr mAb confirmed equal loading (18B). The data are representative of three independent experiments.

FIGS. 19A and 19B. Induction of EGFR-CD533 protein in stably transfected MDA-EGFR-CD533 cells after Dox administration in vitro (48 hr) and in vivo (90 h; see Methods); Western blots using anti-EGFR mAb (19A). Dox induced expression of EGFR-CD533 in vivo resulted in a nearly 3-fold reduction in radiation-induced activation of EGFR within 10 min of a single 4 Gy exposure in MDA-CD533 xenografts; immunoblots of immunoprecipitated EGFR with an anti-phospho-Tyr mAb (19B). Immunoblots of EGFR protein with an anti-EGFR mAb confirmed equal loading (19B, bottom panel). The data are representative of three independent experiments.

FIGS. 20A and 20B. Induction of EGFR-CD533 after intratumoral delivery of Ad-EGFR-CD533 (10¹⁰ pfu). EGFR, EGFRvIII and EGFR-CD533 levels in vivo from frozen tumor tissue relative to in vitro transduction (20A) and their expression levels after transfer of isolated cells to tissue culture and maintenance for 96 h (Ad-LacZ vs. Ad-EGFR-CD533; 20B); Western blots using anti-EGFR mAb.

FIG. 21. Optimization of intratumoral infusion of Ad into MDA xenografts. EGFR-CD533 expression in vivo results in tumor radiosensitization after irradiation at 3×1.5 Gy in vivo. Different administration methods of intratumoral Ad-LacZ infusion to optimize Ad transduction efficiency quantified by x-gal staining of MDA single cell suspensions cells liberated from tumors 48 h after the last Ad infusion. The infusion was modified by infusing the same quantity of Ad vector (10¹⁰ pfu) into 8-10 mm tumors using a single 4- or single 6-track administration or two 6-track infusions on two consecutive days (see Methods). Data represent the mean rate of β-gal positive cells±S.D. from three independent experiments.

FIG. 22. Schematic depiction of the treatment schedule for experiments testing in vivo radiosensitizing effects after intratumoral infusion of Ad-EGFR-CD533 using an MDA xenograft model. The two 6-track infusions were performed on two consecutive days, followed by 3×1.5 Gy on days 4-6, and tumor harvesting and generation of single cells for colony formation assays 24 h after the last irradiation.

FIG. 23. Radiosensitization of MDA-EGFR-CD533 and MDA cells after irradiation of 3×1.5 Gy in vivo under conditions of EGFR-CD533 expression using an ex vivo colony formation assay. Expression of EGFR-CD533 was induced in stably transfected MDA-EGFR-CD533 cells by twice-daily intraperitoneal administration of 300 μg at 12 h intervals for 90 h. The expression of EGFR-CD533, verified by immunoblotting for each experiment, resulted in a significant 38% reduction in ex vivo clonogenic survival relative to the control cells after correcting for plating efficiency (DER=1.6; P<0.0001). The data represent 4 independent experiments. Expression of EGFR-CD533 in MDA cells was induced after in vivo infusion of Ad-EGFR-CD533 using the 2×6-track technique (FIG. 22). Relative to tumor cells transduced with the control vector Ad-LacZ, EGFR-CD533 expression resulted in a significant 46% reduction in ex vivo clonogenic survival, after correcting for plating efficiency. (DER=1.85; P<0.0001). Data represent 3 independent experiments (6 animals per group).

FIG. 24. Western blot analysis of the expression levels of EGFR and ErbB-2 in U-87 MG and U-3 73 MG cells compared to the squamous carcinoma cell line A-43 1. Cell lysates for each cell line were size fractionated by SDS-PAGE and immunoblotted with mA-B specific for EGFR and ErbB-2. Protein standard assays were performed to control for equal loading of protein.

FIG. 25A-B. Radiation-(25A) and EGF-(25B) induced EGFR Tyr-P at specified times after radiation exposure of 2 Gy or 10 ng/ml of EGF in U-87 MG and U-373 MG cells. Cell lysates were immunoprecipated with anti-EGFR mAb and size fractionated by SDS-PAGE; Western blots were reacted with anti-phospho-Tyr-mAb, and the reaction was quantified by chemiluminescence and densitometric scanning. The corresponding values of relative phospho-Tyr levels represent increases over their negative control. Values in 25A are representative of three independent experiments and are given as mean±SD.

FIG. 26. U-373 MG cells were transduced at increasing MOI with LacZ expression recombinant Adenovirus (AdLacZ) to determine the efficiency of transduction. The percent transduction was determined by counting 500 cells at each level of transduction and verifying the number of stained cells versus unstained cells. For further experimental settings, 3 MOI was chosen for U-373 MG cells and 10 MOI for U-87 MG cells respectively with an overall transduction rate >85%.

FIG. 27A-B. Clonogenic survival of gliomas after varying doses of single radiation exposure. Radiation-induced EGFR Tyr-P is blocked by EGFR-CD533 expression. U-87 MG and U-373 MG cells were transduced with either control AdLacZ (27A) or Ad-EGFR-CD533 (27B) with an MOI of 10 or 3 respectively and 48 hours later exposed to 2 Gy radiation. Cell lysates were immunoprecipitated with anti-EGFR mAb and size fractionated by SDS-PAGE; Western blots were reacted with anti-phospho-Tyr mAb, and the reaction was quantified by chemiluminescence and densitometric scanning.

FIGS. 28A and 28B. Clonogenic survival of gliomas after repeated radiation exposures of 2 Gy for three consecutive days. The decrease in relative basal EGFR Tyr-P levels in Ad-EGFR-CD533 transduced cells compared to control AdLacZ transduced cells for cell lines U-87 and U-373 (28A). FIG. 28B: in parallel, cell lysates from the samples shown in 27A which have been immunoprecipitated with anti-EGFR mAb, size fractionated by SDS-PAGE and immunoblotted with a C-terminal binding anti-EGFR mAb.

FIGS. 29A and 29B. Clonogenic survival rate of gliomas after a single radiation exposure of 4 Gy in U-87 MG and U-373 MG cells. Western blot analysis of the expression levels of EGFR and EGFR-CD533 radiation was performed as in FIG. 27. Cell lysates were size fractionated by SDS-PAGE and immunoblotted with a mAb cocktail containing antibodies which react with both COOH— and NH₂-terminal domains of EGFR.

FIG. 30. Radiosensitization of U-87 MG tumor xenografts after infusion of Ad-EGFR-CD533. Clonogenic survival of gliomas after varying doses of single radiation exposure. Cells were either transduced with AdLacZ [⋄] or Ad-EGFR-CD533 [Δ] with an MOI of 10 or left untransduced [¤]. 48 hours later cells were irradiated followed by a further incubation for 24 hours at 37° C. with 5% CO₂ before trypsinization and plating for clonogenic survival. After 12-14 days of incubation, colonies were stained and those with >50 cells were counted to determine the surviving fraction. The log survival was normalized to the number of cells plated, after correcting for plating efficiency. The error bars represent the mean±SEM in three experiments.

FIG. 31. Clonogenic survival after repeated radiation exposures of 2 Gy for three consecutive days. Cells were treated as described in legend of FIG. 29; 48 hours later, cells were irradiated with a single dose of 2 Gy followed by incubation for 24 hours before the next irradiation with 2 Gy. Radiation was performed three times. 24 hours after each radiation exposure, cells were harvested and plated for clonogenic survival. The error bars represent the mean±SEM in three experiments.

FIGS. 32A and 32B. Clonogenic survival rate of gliomas after a single radiation exposure of 4 Gy in U-87 MG and U-373 MG cells. Clonogenic survival rate of U-87 MG (32A) and U-373 MG cells (32B), transduced with either AdLacZ or Ad-EGFR-CD533 compared to untransduced cells after a single radiation exposure of 4 Gy. Survival rate for untransduced cells was set as 100% after correcting for plating efficiency (FIG. 30 legend and Materials and Methods). The additive cell killing effect for the group of Ad-EGFR-CD533 transduced cells compared to control transduced (AdLacZ) or untransduced cells was nearly identical in both cell lines (P<0.0001).

FIG. 33. Radiosensitization of U-87 MG tumor xenografts after infusion of Ad-EGFR-CD533. U-87 MG tumor xenografts with maximum diameters of 8 to 10 mm were treated with recombinant AdLacZ or Ad-EGFR-CD533 delivered intratumorally as described in Materials and Methods. At 72 h post-infusion, animals were irradiated with 3 Gy/day for 3 days at a dose rate of 2 Gy/min using a ⁶⁰Co source. At 24 h post-radiation, tumors were excised, digested to single cell suspension (see Material and Methods for details) and cells were plated for clonogenic survival. Survival rate for AdLacZ infused tumor cells was set as 100% after correcting for plating efficiency (FIG. 29 legend and Material and Methods). The results represent data from two independent experiments performed in quadruplicate; error bars provide±S.E.M. The means are significantly different (t=10.84; p<0.000).

### DETAILED DESCRIPTION OF THE PREFERRED EMBODIMENTS OF THE INVENTION

### DEFINITIONS AND NOMENCLATURE

As used herein, the term “tumor” means a localized growth of cancer cells, which can be the site where a cancer originally formed or can be a metastatic lesion. The terms “tumor cell” and “cancer cell” are used interchangeably herein to mean a malignant cell.

The term “radiosensitize,” when used in reference to a tumor or a tumor cell, means to increase susceptibility of the tumor or tumor cell to the effects of radiation. The term “radiosensitize” is used in a comparative sense and, with regard to the present invention, indicates that the radiation dose to reduce the severity of a cancer in a subject that has been treated as disclosed herein is less than the radiation dose that would have been required if the subject had not been treated.

The term “EGFR” refers to “epidermal growth factor receptor”, a 170 kD transmembrane glycoprotein, contains a growth factor binding site in the extracelluar N-terminal of the molecule and an intracellur tyrosine kinase domain in the C-terminal (Baselga and Mendelsohn, 1994,; Tang et al., 1997).

The terms “EGFR-WT” is used herein to mean a wild-type EGFR.

The term “EGFR-CD533” refers to a truncated EGFR mutant lacking C-terminal 533 amino acids. The EGFR-CD533 lacks mitogenic and transformation activity. It behaves as a dominant-negative mutant, likely through herterodimerization with EGFR-WT to prevent its autophosphorylation (Kashles et al., 1991.; Balabun et al., 1996).

The term “Gy” refers to “gray” the SI unit of absorbed dose, equal to the amount of ionizing radiation absorbed when the energy imparted to matter is 1 joule/kilogram.

The term “accelerated proliferation” or “accelerated repopulation” refers to the radiation-induced proliferation of surviving tumor cells after ionization.

The abbreviations used in this invention are as follows: β-gal, β-galactosidase; Ad, Adenovirus; Ad-EGFR-CD533, Adenovirus containing dominant negative EGFR-CD533; AdLacZ, Adenovirus expressing the bacterial LacZ reporter gene; D37, radiation dose for a survival of 37%; DN, dominant negative; Dox, doxycycline; EGFR Tyr-P, EGFR Tyr phosphorylation; GF, growth factor; JNK1, c-jun NH₂-terminal kinase; mAb, monoclonal antibody; MAPK, mitogen-activated protein kinase; MCC, mammary carcinoma cells; MOI, multiplicity of infection; MTT, 3-(4,5-dimethylthiazol-2-yl)-2,5-diphenyl tetrazolium bromide; pfu, plaque-forming units; Rad, radiation; RTK, receptor tyrosine kinase; SCC squamous carcinoma cells; SDS-PAGE, sodium dodecyl sulfate polyacrylamide gel electrophoresis; Tyr, tyrosine.

Investigations of the molecular mechanism of accelerated repopulation have indicated that the phenomenon may be critically linked to the activation of the epidermal growth factor receptor (EGFR), also known as ErbB1 (Schmidt-Ullrich et al.,1996; Schmidt-Ullrich et al., 1997; Schmidt-Ullrich et al., 1999). Exposure to ionizing radiation appears to activate EGFR in a manner similar to the activation of EGFR by its natural ligands, epidermal growth factor (EGF) and transforming growth factor α (TGF α). A hallmark of the “activation” of EGFR is phosphorylation of a tyrosine residue of the protein. Activation of EGFR by its natural ligands is normally a beneficial event leading to cellular proliferation, for example, during growth of an organism or during tissue repair. However, when the activation occurs within a tumor cell undergoing radiation, cellular proliferation (i.e. accelerated repopulation) is clearly undesirable. The identification of the activation of EGFR as potential critical mechanism of accelerated repopulation suggests that methodology directed to the disruption of EGFR function may be useful in combating this impediment to successful radiation therapy treatment regimens.

The present invention is based on the discovery that the delivery of DNA encoding a mutant, dominant negative form of EGFR to tumor cells causes radiosensitization of tumor cells, and that this radiosensitization is a two-fold phenomenon. On the one hand, direct radiosensitization of tumor cells in general is provided. In addition, the methods of the present invention overcome the phenomenon of accelerated repopulation by inhibiting the proliferative capacity of tumor cells which develops in response to radiation. Of particular interest, as will be seen in Examples 3 and 4 below, infusion of an expression cassette containing a mutant, dominant negative form of the EGFR receptor, EGFR-CD533, to human carcinoma tumor xenografts and malignant glioma xenografts markedly increases the radiosensitivity of the cancer cells in vivo. Without being bound by theory, the invention appears to work by providing the cancer cells with a “defective” form of EGFR which dimerizes with WT-EGFR. (The active form of EGFR is believed to be a dimer. The resulting dimeric protein is non-functional and cannot respond to stimuli (such as normal ligands and radiation) which would otherwise induce activation of the receptor. Activation of the receptor and subsequent cellular proliferation is thus precluded.

Accordingly, the present invention provides a method for 1) directly radiosensitizing cancer cells to radiation, and 2) suppressing the phenomenon of accelerated repopulation in cancer cells of a patient undergoing radiation therapy. One beneficial aspect of the present invention is that the amount of radiation which must be administered in order to destroy a tumor may be decreased. The decrease may be in the frequency and/or the duration of radiation treatments, as well as in the total amount of radiation which is delivered. This is highly beneficial to patients since fewer radiation side effects would be experienced.

In one embodiment of the present invention, the practice of the present invention is carried out in conjunction with a course of radiation therapy. By “in conjunction with” we mean that the delivery of a nucleic acid encoding an altered form of EGFR is carried out in coordination with the radiation treatments. The timing and sequence of radiation treatments and delivery of the nucleic acid may be tailored to the individual needs of the patient and are best determined by skilled practitioners such as physicians. For example, the nucleic acid may be administered prior to, during, after or interspersed throughout a course of fractionated radiotherapy. The nucleic acid may be delivered only once, or several times. Factors such as the age, weight, gender, general physical health, etc. of the patient, the type of cancer being treated, and the like will all contribute to the design of the treatment regimen. Further, the methods of the present invention may be used in conjunction with other therapies such as radiotherapy, chemotherapy, dietary supplementation, and the like. The methods of the present invention may be utilized in conjunction with any suitable cancer therapy in which it is desirable to suppress accelerated repopulation of tumor cells.

The method of the present invention involves delivering to cancer cells a nucleic acid encoding a mutant form of EGFR protein. In a preferred embodiment of the present invention, the mutant form of the EGFR protein is EGFR-CD533, a dominant negative mutant. However, those of skill in the art will recognize that nucleic acids encoding other mutant forms of the EGFR protein may also be used in the practice of the present invention. For example, EGFR-CD33 is a truncated mutant of EGFR which is missing 533 amino acids from the carboxy terminus of the protein. Other truncation mutants which are missing more or less of the carboxy terminal region of EGFR, or mutants which are missing portions of other regions of the protein (e.g. various deletion mutants), may also be employed in the practice of the present invention. Further, mutants resulting from various point mutations, substitutions, frame shift mutations, insertions, and the like, all of which are familiar to those of skill in the art, may be utilized in the practice of the present invention. Nucleic acid encoding any mutant form of EGFR that, upon delivery to cancer cells serves to radiosensitize the cancer cells, may be utilized in the practice of the present invention. Further, those mutant forms may be the result of mutations which occur in nature, or they may be the result of deliberate genetic engineering of EGFR in the laboratory. The mutant forms may or may not be “dominant negative”. Other potential modifications include: the construction of a chimeric protein which includes a mutant form of EGFR together with, for example, a detectable marker protein, but which still functions to radiosensitize cancer cells; or the addition of targeting sequences to the mutant form of EGFR; and the like.

The present invention provides a method for directly radiosensitizing cancer cells, and for disrupting accelerated repopulation by delivering a nucleic acid that encodes a mutant form of the EGFR protein. In a preferred embodiment of the present invention, the nucleic acid is a DNA molecule. However, those of skill in the art will recognize that the nucleic acid may also be an RNA molecule. Further, the nucleic acid may be produced by any of the molecular biology/genetic engineering methods which are well-known to those of skill in the art, e.g. by the growth and maintenance of producer viruses within bacterial or other hosts. Alternatively, the nucleic acid may be synthetically produced by any of the many well-established methods which are well-known to those of skill in the art.

In a preferred embodiment of the present invention, the mutant form of EGFR is EGFR-CD533, the sequence of which is known and publically available, for example, Gene Bank Accession Nos. M38425, K03193, and U48722. However, the practice of the present invention is not confined to the use of the exact sequence of EGFR-CD533, neither of the protein itself or of the nucleic acid encoding the protein as described herein. Those of skill in the art will recognize that many non-substantial modifications of the sequence of the protein and/or the nucleic acid encoding the protein may be made, and all such modifications are intended to be within the scope of the present invention. By “non-substantial” we mean modifications which do not substantially alter the identity and function of the nucleic acid or the protein it encodes. For example, single or multiple base changes (which may or may not alter the protein sequence) may be made in the nucleic acid sequence, or certain chemical modifications may be introduced, in order to, for example, confer added stability to the nucleic acid. Such changes might affect, for example, the in vivo half life of the molecule, or somehow aid in the delivery of the molecule by, for example, resulting in more favorable interactions between the nucleic acid and other components of the delivery system. All such modifications of EGFR-CD533 are intended to be within the scope of the present invention.

In a preferred embodiment of the present invention, the nucleic acid encoding a mutant form of EGFR is DNA which has been inserted into an adenoviral vector for delivery as disclosed herein. However, those of skill in the art will recognize that many other gene delivery systems already exist or are currently under development. Many suitable methods of delivering a nucleic acid to cancer cells are well-known to those of skill in the art and may be utilized in the practice of the present invention. For example, other vectors (both viral and non-viral) may be utilized (e.g. plasmids, viral particles, baculovirus, phage, phagemids, cosmids, phosmids, bacterial artificial chromosomes, viral DNA, P1-based artificial chromosomes, yeast plasmids, and yeast artificial chromosomes., and the like. (See, for example, Kirshenbaum et al., 1993). Some forms of viral vectors may be especially useful (e.g. viral vectors such as retrovirus, adenovirus or adenovirus-associated vectors). Alternatively, the nucleic acid may be delivered via liposomes or liposome-type delivery systems, or via attenuated bacterial delivery systems, by binding (either covalently or non-covalently) to another molecule which enhances delivery, by direct injection of the nucleic acid in or around the tumor, or by catheterization, and the like. Further, other procedures which enhance the delivery of nucleic acids into cells may be utilized in conjunction with the practice of the present invention, e.g. various means of altering cell membrane permeability (e.g. ultrasound, exposure to chemicals or membrane permeability altering substances, and the like). Any appropriate means of delivery of the nucleic acid may be utilized in the practice of the present invention so long as the delivery results in radiosensitization of cancer cells.

In a preferred embodiment of the present invention, the nucleic acid is delivered via a replication-deficient adenoviral vector, Ad-EGFR-CD533. Those of skill in the art will recognize that various modifications of the vector may be made without substantially altering its character or ability to function in the practice of the present invention. All such modifications are intended to be encompassed within the scope of the present invention. For example, changes may be made with respect to the precise location of the encoding DNA within the vector, or to the nature of the promoter. In a preferred embodiment, the expression of EFGR-CD533 is under the control of doxycycline (DOX)-inducible promoter (Contessa et al, 1999; Gossen et al. 1995). In addition, other vector elements which impact on the expression of the EGFR mutant may be added or altered. For example, elements such as enhancers, various inducible elements, and the like may be added or altered. All such variations are intended to be within the scope of the present invention.

The present invention also provides a therapeutic agent comprising an effective dose of a nucleic acid encoding a mutant form of EGFR and a suitable carrier. The nucleic acid to be utilized in the practice of the present invention may conveniently be provided in the form of formulations suitable for administration to mammals. A suitable administration format may best be determined by a medical practitioner for each patient individually. Suitable pharmaceutically acceptable carriers and their formulation are described in standard formulations treatises, e.g., Remington's Pharmaceuticals Sciences by E. W. Martin. See also Wang, Y. J. and Hanson, M. A. “Parental Formulations of Proteins and Peptides: Stability and Stabilizers”, Journals of Parental Sciences and Technology, Technical Report No. 10, Supp. 42:2S (1988). Vectors of the present invention should preferably be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions may also be prepared to enhance shelf life and stability. The therapeutically useful compositions for use in the practice of the invention are prepared by mixing the ingredients following generally accepted procedures. For example, the selected components may be mixed to produce a concentrated mixture which may then be adjusted to the final concentration and viscosity by the addition of water and/or a buffer to control pH or an additional solute to control tonicity.

For use by the physician, the compositions will be provided in dosage form containing an amount of a vector of the invention which will be effective in one or multiple doses to induce suppression of accelerated proliferation. As will be recognized by those in the field, an effective amount of therapeutic agent will vary with many factors including the age and weight of the patient, the patient's physical condition, and the type of cancer being treated, and other factors.

The effective dose of the compounds of this invention will typically be in the range of at least about 10⁷ viral particles (where ˜20-100 particles is equal to 1 pfu), preferably about 10⁹ viral particles, and more preferably about 10¹¹ viral particles. The number of viral particles may, but preferably does not exceed 10¹³. As noted, the exact dose to be administered is determined by the attending clinician.

The delivery of the nucleic acid may be accomplished by a variety of methods. In a preferred embodiment, a vector encoding the mutant EGFR is delivered via infusion. However, those of skill in the art will recognize that delivery may also be effected by any of a number of other routes. For example, the vector may be injected, or delivery may be carried out by any of several techniques, e.g. intratumoral injection, positive pressure, and continuous flow infusion. Or, delivery may be systemic and may be accomplished by, for example, oral or intravenous administration, or by inhalation. Any delivery means may be utilized in the practice of the present invention so long as delivery of the nucleic acid results in the suppression of the accelerated repopulation of the cancer cells.

As described herein, nucleic acid encoding a mutant form of EGFR is administered in concert with radiation therapy. Determination of the dose of radiation to be utilized in radiotherapy treatment is well known in the art. Administration of radiotherapy as fractionated doses over a period of time can provide advantages over administration of a single large dose. However, the method of the present invention may be practiced with any suitable radiotherapy regimen. As disclosed herein, the clinically relevant dose of about 2 Gy is preferred in this invention.

The present invention provides methods for the radiosensitization of cancer cells, both direct radiosensitization and the disruption of accelerated repopulation. In one aspect of the present invention, the cancer cells are those in which EGFR is expressed. In a preferred embodiment of the present invention, radiosensitization of cancer cells of mammary tumor and glioma origin is effected. This is based on the results given in the Examples below, which demonstrate that expression of an EGFR mutant results in radiosensitization of human mammary carcinoma cells and glimoma cells both in vitro and in vivo. However, those of skill in the art will recognize that the methods of the present invention may be practiced with respect to a wide variety of cancer types including but not limited to squamous carcinoma, mammary, malignant glioma, cervical, prostate, head and neck cancer and the like. Since the proliferative pathway in which EGFR participates is ubiquitous, it is highly likely that the method of the present invention would have widespread applicability with respect to the suppression of accelerated repopulation, regardless of tumor origin. For example, human squamous carcinoma cells appear to undergo accelerated repopulation via a similar mechanism (Dent et. al., 1999). Any type of cancer cell which responds to the methods of the present invention may be radiosensitized by the methods of the present invention.

The data presented in Examples 1-4 below provides evidence that the EGFR receptor can be effectively targeted with a genetic therapy approach resulting in tumor cell radiosensitization.

Example 1 describes investigations concerning the effects of the inducible expression of a dominant-negative EGFR mutant (EGFR-CD533) which lacks mitogenic and transformation activity on radiation-induced activation of the EGFR and critical cytoprotective responses, such as MAPK activation. The results demonstrate that this genetic approach of disrupting EGFR function can result in a significant enhancement of radiation toxicity (Contessa et al. 1999).

Example 2 describes data which demonstrates that both MAPK and JNK1 activation in response to radiation occur through EGFR-dependent and -independent mechanisms. Data in Example 2 also demonstrates that radiation-induced activation of EGFR results in downstream activation of MAPK, which may affect the radiosensitivity of carcinoma cells (Reardon et al., 1999).

Example 3 describes a cancer genetic therapy study and demonstrates that disruption of EGFR function by over-expression of the dominant-negative EGFR mutant (EGFR-CD533) causes radiosensitization of human mammary carcinoma cell both in vitro and in vivo. (Lammering et al., a, submitted).

Example 4 describes an investigation of the radiation-induced activation of EGFR in human malignant glioma cells. The results show that inhibition of EGFR function via gene transfer of the dominant-negative EGFR mutant (EGFR-CD533) to glioma cells both in vitro and in vivo causes radiosensitization of the cancer cells (Lammering et al., b, submitted).

It will be apparent to one skilled in the art that various substitutions and modifications may be made to the invention disclosed herein without departing from the scope and the spirit of the invention. Accordingly, the following examples are intended to illustrate but not limit the present invention.

### EXAMPLES

### Example 1

### The Inducible Expression of Dominant Negative EGFR-CD533 Results in Radiosensitization of Human Mammary Carcinoma Cells

### Example 1

### Materials and Methods

Reagents. Unless specified otherwise all reagents were from Sigma Chemical Co. (St. Louis, Mo.). The immunochemical reagents have been described (Schmidt-Ullrich et al., 1996; Kavanagh et al., 1995; Carter et al.,1998) except for the following: A mAb cocktail reacting with both C- and N-terminal domains of EGFR (Ab14, Neo Markers, Fremont, Calif.) was used for Western blotting of EGFR. mAb E12120 (Transduction Laboratories, Louisville, Ky.) which reacts specifically with autophosphorylated EGFR was used for probing blots for activated EGFR. ErbB-2 was immunoprecipitated and blotted with rabbit polyclonal Ab #sc-284 (Santa Cruz Biotechnology, Santa Cruz, Calif.). Radioisotopes were from Dupont-New England Nuclear (Boston Mass.). Tetracycline-free fetal bovine serum (FBS) was from Clontech (Palo Alto, Calif.) and RPMI 1640 (RPMI) from Gibco (Gaitherburg, Md.).

Construction and characterization of cell lines. The parental MCC lines, MCF-7 and MDA-MB-231, have been described (Schmidt-Ullrich et al., 1997; Kavanagh et al., 1998). For optimal efficiencies, transfections of MCF-7 or MDA-MB-231 cells were performed by the calcium phosphate and electroporation methods, respectively (Sambrook et al., 1989). Stable clones expressing the tetracycline resistance transactivator were selected after transfection with pUHD172-1neo (provided by H. Bujard, University of Heidelberg, Germany) and screened by transient transfection with the reporter plasmid, pUH13-3, which contains the tet operator and the luciferase reporter gene (Gossen et al., 1995). The pRK5HerNa8 plasmid encoding the EGFR cDNA, lacking the C-terminal 533 amino acids (Balaban et al., 1996; provided by A. Ullrich, Max-Planck-Insitut fuer Biochemie, Martinsried, Germany), was cloned as a 1.75 kb XbaI fragment into pUHC10-3 containing the tet operator to yield the plasmid, pUHC10-3-EGFR-CD533. To obtain EGFR-CD533 expressing cells, the MCF7-TR5 and MDA-TR15 clones from the first level of transfections were co-transfected with this plasmid and pRSVhygro, 20:4 μg/10 cm dish, and selected for resistance to hygromycin at 200 g/ml. Vector control cell lines, MDA-TR15-Luc27 and MCF-TR5-Luc24, were obtained by co-transfection with pUHC10-3 (Gossen et al., 1995) containing the luciferase reporter gene and pRSVhygro.

Induction of EGFR-CD533 was quantified at the level of mRNA and protein after 48 h induction by Dox at 1 μg/ml (Gossen et al., 1995). The cytoplasmic mRNA levels in MCF-TR5-EGFR-CD533 cells were quantified by RNase protection, and Northern analysis was employed for MDA-TR15-EGFR-CD533 cells using established procedures (Schmidt-Ullrich et al. 1994). The kinetics of EGFR-CD533 induction and its decay after Dox removal were monitored by metabolic labeling with ³⁵S-Met as described in the text.

The possible effects of EGFR-CD533 expression on the protein levels of EGFR-WT and erbB-2 were examined by Western blotting of cell lysates or immune precipitates (Schmidt-Ullrich et al., 1997). Tyr autophosphorylation of EGFR was monitored as previously described (Schmidt-Ullrich et al., 1996) with the following modifications. Dox at 1 μg/ml was added 48 h prior to radiation exposure and cell harvesting with a medium change to RPMI/0.5% FBS with or without Dox for the final 24 h. This medium was replaced by RPMI/0.5% FBS 2 h prior to treatment. Under these conditions, cell cultures were at 70-80% confluence and experimental treatments were initiated, such as irradiation with ⁶⁰Co at a dose rate of 1.7 Gy/min at 20° C. or exposure to 2 ng/ml of EGF for 5 min. MAPK assays were carried out identically to procedures described for EGFR Tyr phosphorylation followed by immune complex assays (Carter et al. 1998; Kavanagh et al.).

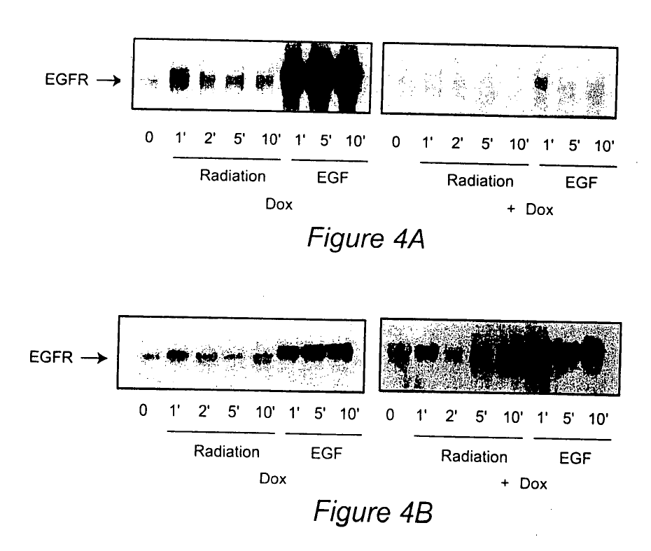

Besides using single radiation dose response analyses (Elkind and Sutton. 1960), a scheme of repeated radiation exposures combined with a MTT cell growth assay has been devised (see FIG. 4 for schematic depiction; Schmidt-Ullrich et al. 1997; Kavanagh et al., 1995). This assay was specifically designed to investigate interactions between repeated radiation exposures and cell proliferation and was applied to demonstrate the effect of EGFR-CD533 expression on cell survival. MDA-TR15-EGFR-CD533 cells were seeded in RPMI/5% FBS at 1000 or 2000 cells/well in 96-well tissue culture plates for control and radiation experiments, respectively. After cell attachment overnight, 50% of the cultures were exposed to Dox at 1 μg/ml. Within 24 h of Dox exposure, drug-exposed and control cultures were irradiated once-daily with 2 Gy for 5 consecutive days. Without additional feeding, cell growth was assessed on days 1, 4, and 7 after irradiation using the MTT assay (Schmidt-Ullrich et al. 1997; Kavanagh et al., 1995).

### Example 1

### Results

Induction of EGFR-CD533 mRNA and protein. For studies on the mechanisms and the modulation of radiation-induced cell proliferation, a dominant negative EGFR mutant, EGFR-CD533, was chosen and expressed, for the first time, in human carcinoma cells (Redemann et al., 1992). The dependence of autocrine growth-regulated carcinoma cells on EGFR-WT function also dictated the use of an inducible expression system because of potential toxic effects under conditions of constitutive expression. The two plasmid tetracycline-on expression system was employed (Gossen et al., 1995). From the initial transfection with pUHD 172-1neo, the MCF-TR5 and MDA-TR15 clones were chosen based on the extent of inducible EGFR-CD533 expression as quantified by the luciferase reporter plasmid, pUHD13-3. These cloned cell constructs were then co-transfected with pUHC10-3-EGFR-CD533 and pRSVhygro and yielded the stable clones MCF-TR5-EGFR-CD533 and MDA-TR15-EGFR-CD533.

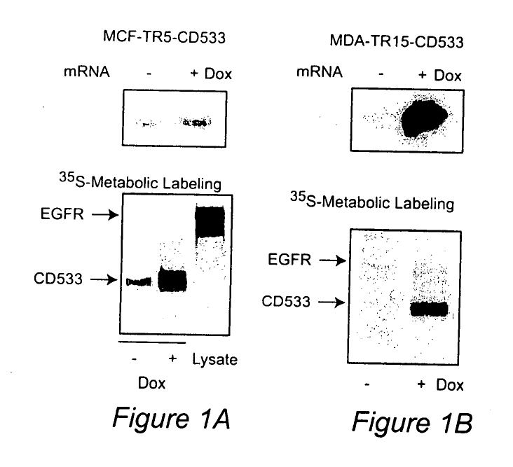

The MCF-TR5-EGFR-CD533 cells produced a 5-fold increase in EGFR-CD533 mRNA levels after 48 h of Dox exposure (FIG. 1A). This translated into a greater than 20-fold induction of the 110 kD EGFR-CD533 truncated receptor. On the other hand, the MDA-TR15-EGFR-CD533 cells demonstrated a greater than 30-fold increase in EGFR-CD533 mRNA levels (FIG. 1B) and a greater than 50-fold induction at the protein level. Because of the higher EGFR-WT expression levels in MDA-MB-231 cells, MDA-TR15-CD533 cells were selected for illustrating the responses described below.

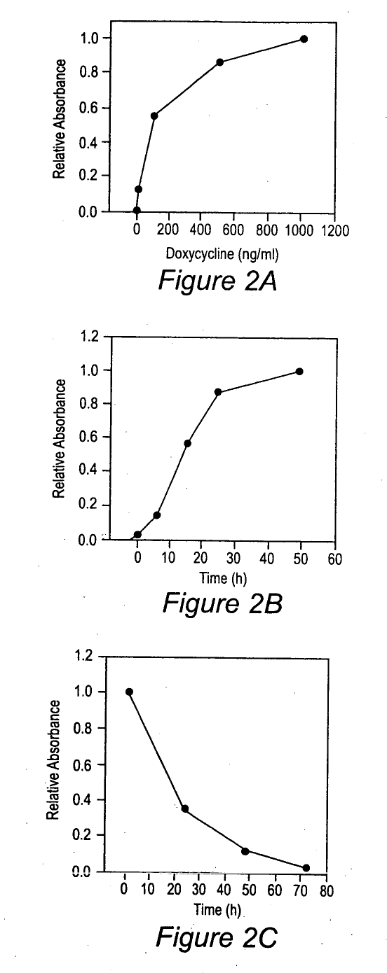

Dose dependence and kinetics of EGFR-CD533 induction by Dox. The induction and decay kinetics of EGFR-CD533 protein were examined. At 48 h exposure to Dox between 0.01 and 1.0 μg/ml, a half maximal response was seen at 0.1 μg/ml which is consistent with previous reports (FIG. 2A). In studying the induction kinetics of EGFR-CD533, the fastest rate of induction at 1.0 μg/ml Dox measured by ³⁵S-Met incorporation occurred between 8 and 24 h at which time the EGFR-CD533 had been induced by >90% (FIG. 2B). Thereafter, the rate leveled off substantially and maximum induction was achieved by 48 h. The decay kinetics were measured after 48 h induction followed by Dox removal. A 65% reduction of EGFR-CD533 was seen after 24 h reaching 90% at 48 h (FIG. 2C). The studies illustrated were obtained with MDA-TR15-EGFR-CD533 cells, but were found to be identical to those for MCF-TR5-EGFR-CD533 cells (data not shown). The relatively fast induction and decay kinetics make for an attractive experimental system in studying the effects of EGFR-CD533 on radiation-mediated cellular responses.

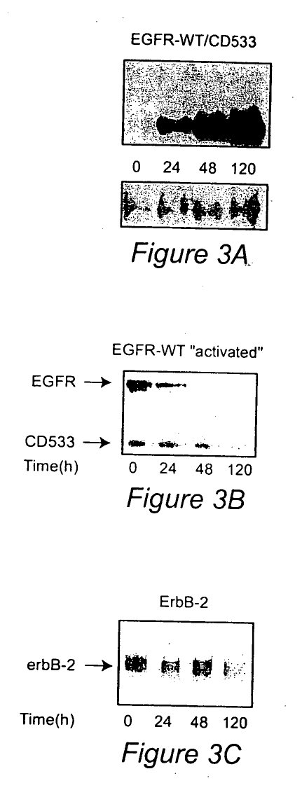

Effects of EGFR-CD533 on expression and activation of EGFR-WT. It was important to examine whether the expression of EGFR-CD533 affected the expression levels or the function of EGFR-WT. Using a mixture of mAbs (Ab14) against the N-terminal portion of the protein, a Western blot analysis of immunoprecipitated EGFR-WT and EGFR-CD533 indicated that even with a robust induction of EGFR-CD533, EGFR-WT protein expression levels remained essentially constant with a suggested minimal decline at 120 h (FIG. 3A). Expression of other erbB family members of MDA-MB-231 cells (Lupu and Lippman, 1993), including erbB-2 shown in FIG. 3C, was also unaffected by EGFR-CD533 expression except at later timepoints of induction. However, when a mAb specific for the autophosphorylated form of EGFR was used in a Western blot analysis of immunoprecipitated EGFR-WT and EGFR-CD533, there was a time-dependent marked effect on Ab reactivity (FIG. 3B); within 48 and 120 h of Dox exposure, the expression of autophosphorylated EGFR-WT was reduced to 40 and <10%, respectively, of the control. These studies indicate that expression of EGFR-CD533 has only marginal effects on EGFR-WT expression but interferes with receptor activation.

It has previously been demonstrated that EGFR becomes rapidly activated as assessed by Tyr phosphorylation after radiation exposures in the 0.5 to 5 Gy dose range, and that this activation can be linked to a proliferation response in A431 SCCs (Schmidt-Ullrich et al. 1996; Schmidt-Ullrich et al. 1997; Kavanagh et al., 1995). The results shown in FIG. 4A (left eight lanes) demonstrate that MDA-TR15-EGFR-CD533 cells responded to 2 Gy of radiation and exposure to 2 ng/ml of EGF with EGFR-WT activation in a similar way as had been shown for the parental MDA-MB-231 cells (Schmidt-Ullrich et al. 1996). In the absence of Dox, MDA-TR15-EGFR-CD533 cells showed a 3.8-fold increase in EGFR-WT Tyr phosphorylation within 1 min of radiation exposure which decreased to baseline activity within 10 min. In comparison, 2 ng/ml of EGF induced a 9.8-fold activation that maximized at about 5 min. Importantly, these responses were completely ablated in MDA-TR15-EGFR-CD533 cells under conditions of maximum EGFR-CD-533 expression (FIG. 4A, right eight lanes). The expression of the luciferase control vector did not affect the radiation-induced activation of EGFR-WT (FIG. 4B). This data demonstrates that the expression of EGFR-CD533 converted autocrine growth regulated MCCs into functionally dominant negative EGFR mutants.

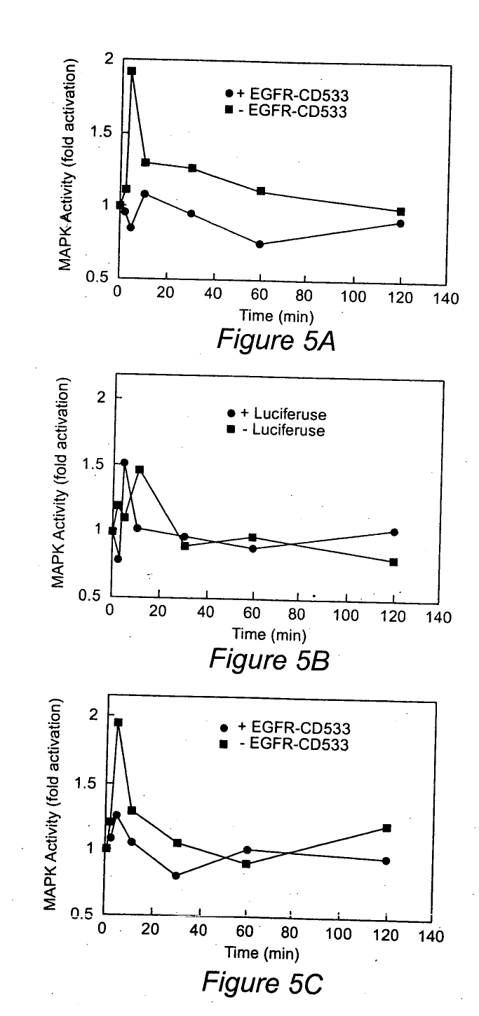

Effects of EGFR-CD533 expression on MAPK responses to radiation exposure. The effects of EGFR-CD533 expression on MAPK activity were examined because MAPK represents a critical link between EGFR activation and cellular proliferation responses induced by ionizing radiation (Schmidt-Ullrich et al. 1997). The responses of MAPK were examined over 120 min for MDA-TR15-EGFR-CD533 cells after exposure to 2 Gy with and without prior exposure to Dox. Radiation-induced an immediate, approximately 2-fold activation of MAPK as demonstrated in FIG. 5A. This MAPK response was completely inhibited after expression of EGFR-CD533. The responses were not due to the transfection or Dox exposure, since cells stably transfected with the luciferase control vector, in place of EGFR-CD533, demonstrated a radiation-induced MAPK response irrespective of Dox exposure (FIG. 5B). A similar radiation induced MAPK response was observed with MCF-TR5-EGFR-CD533 cells (FIG. 5C). These results demonstrate that EGFR function is essential for radiation-induced activation of MAPK and suggest that disruption of this cytoprotective pathway may allow the modulation of cellular radiation responses.

Effect of EGFR-CD533 expression on cell proliferation after repeated radiation exposures. To assess the possible effect of EGFR-CD533 expression the radiosensitivity of MCCs, single radiation dose-response clonogenic survival analyses between 1 and 8 Gy were performed (Elkind and Sutton, 1960). Neither MCF-TR5-EGFR-CD533 nor MDA-TR15-EGFR-CD533 cells exhibited significant differences in radiosensitivity. The values of mean inactivation doses \overline{D} (Fertil et al. 1984) for the MDA-TR15-EGFR-CD533 cells with or without Dox exposure were 1.72 and 1.79, respectively, and for the MCF-TR5-CD533 cells, 1.69 and 1.54, respectively. Both data pairs were not significantly different.

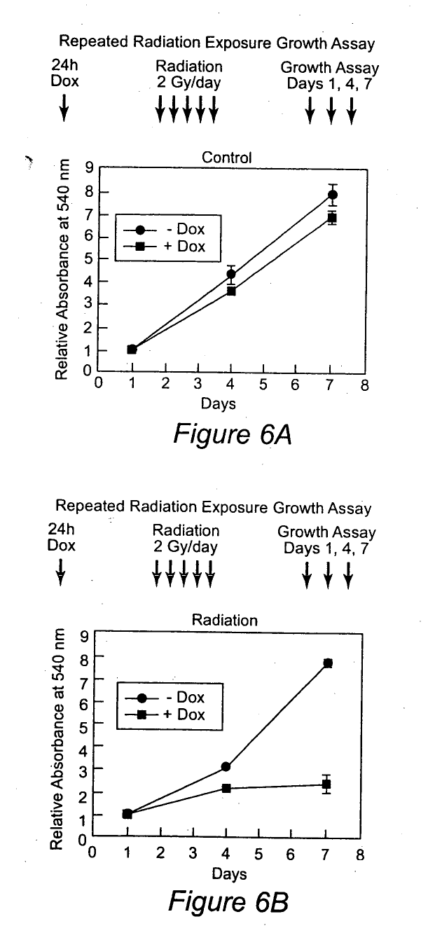

In an attempt to amplify potential interactions between radiation toxicity and the growth inhibitory effects of EGFR-CD533 expression, repeated radiation exposure experiments were designed. Because of relatively low cell numbers, the MTT assay did not discern significant differences between the four combinatorial conditions of EGFR-CD533 expression and irradiation on day 1 of the 7-day growth assay (FIG. 6). Over 7 days, the un-irradiated cell cultures grew at similar rates irrespective of EGFR-CD533 expression with a modest but significant growth retardation of the MDA-TR15-EGFR-CD533 cells under conditions of EGFR-CD533 expression (FIG. 6A). In contrast, expression of EGFR-CD533 markedly affected the growth of MDA-TR15-EGFR-CD533 cells after 5 daily 2 Gy radiation exposures (FIG. 6B). Without the expression of EGFR-CD533, the irradiated cells resumed proliferation rates on days 4 and 7 that were similar to or even exceeded those of the un-irradiated control cultures. In contrast, the cultures irradiated under conditions of EGFR-CD533 expression demonstrated significant growth retardation which was reflected in significantly reduced cell numbers on days 4 and 7 (FIG. 6B). Therefore, on day 7 of the growth period, MDA-TR15-EGFR-CD533 cells expressing EGFR-CD533 during and after irradiation yielded a minimum 4-fold lower cell numbers.

These results illustrate that EGFR-WT function is required for carcinoma cell proliferation responses after repeated radiation exposures, and that the expression of EGFR-CD533 enhances radiation toxicity.

### Example 2

### Dominant Negative EGFR-CD533 and Inhibition of MAPK Modify JNK1 Activation and Enhance Radiation Toxicity of Human Mammary Carcinoma Cells

### Example 2

### Materials and Methods

Cell lines and reagents. Mammary carcinoma cell lines, MDA-TR15, MDA-TR15-EGFR-CD533 and MDA-TR-Luc used in this study were developed in our laboratory and derived from the MDA-MB231 cell line (See Example 1). The cell line MDA-TR15, was used to generate both MDA-TR15-EGFR-CD533 and MDA-TR15-Luc cell lines and was used as a negative control in the study. Treatment of MDA-TR15-EGFR-CD533 and MDA-TR15-Luc cells with 1 μg/ml Dox for 48 h induced maximum expression of dominant negative EGFR-CD533 or luciferase (Example 1). Recombinant adenovirus expressing Tam 67 (Ad-TAM 67) (Brown et al., 1993; Auer et al., 1998), an NH₂-terminally truncated c-Jun which functions as a dominant negative mutant of Jun/AP-1, was obtained from Dr. Micheal Birrer (National Cancer Institute, Bethesda, Md.).

Antibodies (Abs) to p42 MAPK (ERK2 for IP) and JNK1 (sc-571) were from Upstate Biotechnologies, Inc. (Lake Placid, N.Y., USA) and Santa Cruz Biotechnologies (Santa Cruz, Calif., USA), respectively. Abs to N-terminal c-Jun and Ser-63 phospho-c-Jun were obtained from New England Biolabs (Beverly, Mass., USA) while C-terminal c-Jun antibody (Ab-1) was obtained from Oncogene Research Products (Cambridge, Mass., USA). Myelin basic protein (MBP) as MAPK substrate (Sigma Chemical Co., St. Louis, Mo., USA) was used as a stock of 10 mg/ml in H₂O. PD98059 (Calbiochem, La Jolla, Calif., USA) was prepared as a 10 mm stock in DMSO and stored at −20° C. (Dudley et al., 1995. Proc. Natl. Acad. Sci. USA, 92, 7686-7689). Radiolabeled ³²P-γ-ATP was from NEN-Dupont (Boston, Mass., USA). Protease/phosphatase inhibitors and other chemical reagents were purchased from Sigma Chemical Co., (St. Louis, Mo., USA). Farnesyltransferase inhibitor I (FTI; Calbiochem, La Jolla, Calif., USA) (Manne et al., 1995. Oneogene, 10, 1763-1779) was prepared as a 1 mm stock solution in sterile PBS and stored at −20° C. GST-Jun (aa 1-139) protein expressed from a GST-Jun expression construct was purified on glutathione Sepharose 4B beads (Pharmacia Biotech Inc. Piscataway, N.J., USA) according to manufacturer's instructions.

For TUNEL assays, terminal transferase and fluorescein 12-dUTP (Boehringer Mannheim. Germany) was used with mounting media containing propidium iodide (Vector Laboratories, Inc., Burlingame. Calif. USA). Detection of protein by Western blotting was performed using the Western Star Chemiluminescence kit (BioRad Laboratories, La Jolla, Calif., USA).

Cell treatments and irradiation. All cells were routinely grown in RPM11640 medium (Sigma Chemical Co., St. Louis, Mo. USA) containing 5% tetracycline negative fetal calf serum (ClonTech Laboratories Inc., Palo Alto, Calif., USA), RPMI/5FCS. MDA-TR15 EGFR-CD533 and MDA-TR15 cells were seeded at either 9.0×10⁵ or 1.7×10⁶ cells per 10 or 15 cm plate, respectively (Schmidt-Ullrich et al., 1997). Cells were cultured for a total of 5 days in RPMI/5FCS. On day 3, cells were exposed to RPMI/5FCS with or without 1 μM Dox. On day 4, media was replaced by low serum RPMI/0.5% FCS, with or without Dox for an additional 24 h, followed by equilibration with RPMI/0.5% FCS without Dox 2 h prior to irradiation. For treatment with PD98059, cells were seeded at densities described above and exposed to RPMI/0.5% FCS for 24 h. Thereafter, cells were exposed to RPMI/0.5% FCS containing 10 μM PD98059 2 h prior to irradiation. Cellular Ras function was inhibited by incubating cells for 24 h in 50 μM FTI prior to irradiation. For expression of Tam 67 from recombinant adenovirus cells were routinely infected at an MOI of 100 for 48 h prior to irradiation or immunoblotting.

In all radiation experiments, cells were exposed to 2 Gy of ionizing radiation at a dose rate of 1.2 Gy/min using a ⁶⁰CO source. For time course experiments, cells were irradiated and incubated at 37° C. for the times specified. Thereafter, media was removed and cells were washed once in ice cold PBS. Cells were immediately frozen on dry ice and stored at −20° C. until further processing.

Immunoprecipitation and Western analysis. For immunochemical analyses, cells frozen at −20° C. were lysed in ice-cold lysis buffer, 25 mM β-glycerophosphate, pH 7.4, 25 mM Tris, pH 7.4, 10% (v/v) glycerol, 1.5 mM EGTA, 0.5 mM EDTA, 1% Triton X-100, 1 mM Na-orthovanadate, 1 mM Na-pyrophosphate, 10 μg/ml aprotinin, 10 μg/ml leupeptin, 1 μm benzamidine, 100 μg/ml PMSF. Insoluble material was removed by centrifugation at 14 000 g for 10 min, and the clarified protein lysates were divided equally for immunoprecipitation with antibodies against JNKI and MAPK using established techniques (Boulton et al., 1990). Lysates were incubated with Ab at 4° C. for 3 h, the agarose-Ab immunoprecipitates were washed twice in lysis buffer, once in lysis buffer containing 0.1% Triton X-100, and once in 1× kinase buffer, 25 mM β-glycerophosphate, pH 7.4, 15 mm MgCl₂. For Western blot analyses, equal amounts of cellular protein were separated by SDS-PAGE, electroblotted to nitrocellulose, probed with specific Abs against c-Jun protein and c-Jun 0phosphorylated at Ser-63, followed by chemilumescence detection. Expression of Tam 67 in MDA-TR15-EGFR-CD533 cells infected with recombinant adenovirus was determined by Western analysis using a c-Jun Ab specific for the COOH-terminus.

Immune complex kinase assays. Immunoprecipitates were suspended in 40 μl H₂O containing the appropriate substrates, consisting of 20 μg MBP for MAPK and 10 μg GST-c-Jun (aa 1-139) protein for JNK1. For kinase assays, 10 μl of 5× kinase buffer, 125 mM β-glycerophosphate, pH 7.4, 75 mM MgCl₂, 500 μm ATP, and [³²P]-γ-ATP (5000 c.p.m./pmol) was added to each immunoprecipitate and incubated with periodic vortexing at 37° C. for 30 min.

Phosphorylation of substrate by MARK was measured following substrate binding to P81 assay paper (Wu et al., 1993). Aliquots of the assay mixture were placed on P81 paper and non-specific binding of [³²P]-γ-ATP removed by extensive washing in 180 mM phosphoric acid. Incorporation of ³²P into bound MBP substrate was measured by liquid scintillation spectroscopy (Beckman Instruments Inc, Schaumburg, Ill., USA). For JNK1 assays (Derijard et al., 1994), sample buffer was added to the kinase mixture to stop the reaction, the samples were boiled and separated by SDS-PAGE in 10% polyacrylamide gels. Coomassie stained GST-Jun (aa 1-139) protein was excised from the gel, and ³²P incorporated into the bound substrate was quantified by liquid scintillation spectroscopy.

TUNEL assay for apoptosis. Cells were placed at a density of 2×10⁴ cells into dual well slides and allowed to adhere overnight in RPMI/5FCS. Cells were then exposed, prior to irradiation, for 48 h to Dox or control solute for induction of EGFR-CD533. PD98059 at a final concentration of 10 μM was added 2 h prior to irradiation at 2 Gy. Cells were infected with recombinant Ad-TAM67 for expression of Tam67 for 48 h prior to irradiation. After incubation of cells for an additional 24 h, apoptosis was assessed by TUNEL assay measuring double stranded DNA breaks (Chelliah et al., 1997). Slides were stored at −20° C. until examination by fluorescence microscopy using a fluorescein filter. Approximately 500 cells were counted for each treatment condition, and the results were compiled from three different experiments.

Growth rate analysis. For irradiation and control conditions, 1000 and 500 cells per well of 24-well plates were seeded, respectively. Cells were maintained in RPMI/5FCS without media change throughout the period of the experiment. After 24 h of seeding, 50% of the plates were treated with 10 μM PD98059 for 2 h while negative controls were exposed to equivalent DMSO concentrations of 1 μl/ml in RPMI/5FCS. This was followed by 2 Gy daily radiation exposures for 5 consecutive days; control cells were sham irradiated. Cell growth was determined using the 3-[4,5-Dimethylthiazol-2-yl)-2,5-diphenyltetrazolium bromide (MTT) assay (Denizot and Lang., 1986; Carmicheal et al., 1987) on days 1, 4 and 7 days following the last day of irradiation. Growth rates were quantified relative to absorbance at 540 nm on day 1.

Statistical analysis. The effects of various treatments were compared using one way analysis of variance and a two tailed t-test. Differences with a P-value of <0.05 were considered statistically significant. All values and means shown are+s.e.m. from an average of at least three independent experiments. For analysis of apoptotic data a 2-squared factorial analysis of variance using the standard F-test was implemented for the determination of synergy between radiation and either PD98059 or EGFR-CD533.

### Example 2

### Results

EGFR function and radiation-induced activation of MAPK and JNK1. The MDA-TR15-EGFR-CD533 cell line, a derivative of the parental MDA-MB-231 cell line, expresses dominant negative EGFR-CD533 under the control of a two-plasmid Dox-inducible expression system (Gossen et al., 1995). All experiments examining the role of EGFR in cellular radiation responses were performed without and with expression of the dominant negative EGFR-CD533. The maximum expression of EGFR-CD533 was achieved by exposing cells to 1 μg/ml of Dox for 48 h prior to irradiation. Analyses of signal transduction cascades were limited to immediate responses within 60 min after irradiation since radiation-induced transient activation of EGFR has been defined as a 1-5 min transient increase in Tyr-phosphorylation (Schmidt-Ullrich et al., 1996; Schmidt-Ullrich et al., 1997).

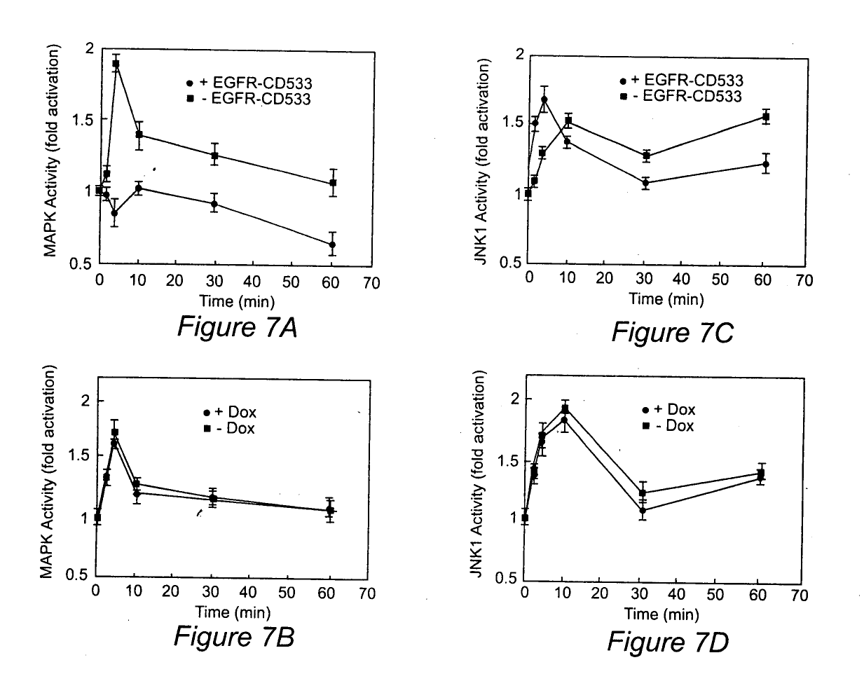

In the absence of EGFR-CD533 expression, the activation of EGFR after 2 Gy of ionizing radiation mediated a maximum 1.9 (±0.04)-fold activation of MAPK within 4 min which returned to base-line levels after 60 min (FIG. 7A). After induction of EGFR CD533 expression, the radiation-induced activation of MAPK was completely inhibited, indicating that this response depended on EGFR function (FIG. 7A).

Radiation-induced activation of JNK1 followed slightly slower kinetics with an early peak of 1.5 (±0.1)-fold stimulation above basal levels at 10 min and a later rise in activity (FIG. 7C) to a 1.6(±0.03)-fold increase at 60 min. The expression of EGFR-CD533 did not block the radiation-induced 1.7(±0.1)-fold activation of JNK1 at the early time points. However, the activation of JNK1 30 min after irradiation was completely ablated by expression of EGFR-CD533 (FIG. 7C). This data suggests that radiation-induced JNK1 activation occurred through both EGFR-dependent and -independent mechanisms.

Although the immediate enhancement of signals significant relative to controls, the specificity of these findings was further corroborated by parallel experiments with vector control cells MDA-TR15. As demonstrated in FIGS. 7B and 7D, the radiation-induced activation of MAPK and JNK1 was not altered by exposure of cells to Dox. Additional control experiments with MDA-TR15-Luc cells, which inducibly express luciferase instead of EGFR-CD533, confirmed that the changes of radiation-induced MAPK and JNK1 activation were specific for EGFR-CD533.

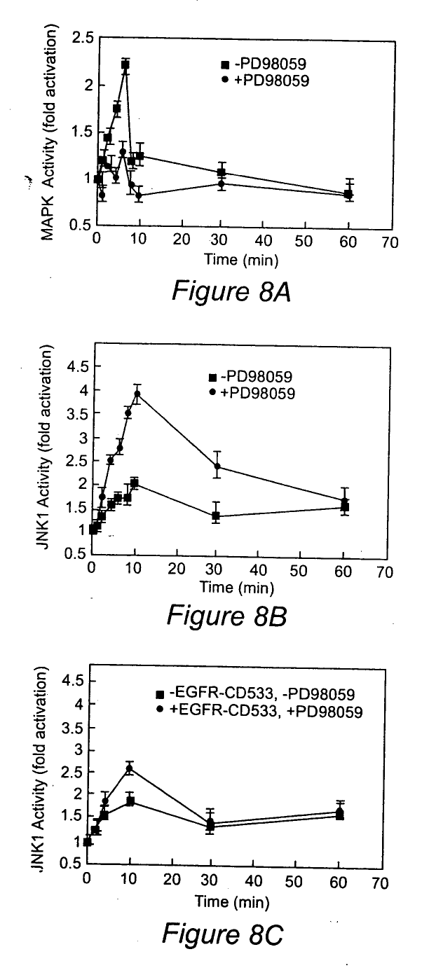

EGFR function as a requirement for the enhanced immediate JNK1 activation under conditions of MAPK inhibition. The ability of radiation to induce JNK1 activation under conditions of preserved EGFR function but with inhibition of the MAPK pathway using the specific inhibitor of MEK1/2, PD98059, was assessed. Data shown in FIG. 8A demonstrates that pretreatment of MDA-TR15-EGFR-CD533 cells with 10 μm PD98059 inhibited >70% of radiation-induced MAPK activation. This inhibition of MAPK resulted in an overall 3.9 (±0.1)-fold increase in the immediate JNK1 response, equivalent to a twofold enhancement of JNK1 activation relative to the 1.9 (±0.1)-fold stimulation with radiation alone (FIGS. 7C and 8B). The difference of these values was statistically significant (P<0.01). The JNK 1 response after 30 min was unaffected.

The relative contributions of EGFR and MAPK to the enhanced radiation-induced JNK1 activation were further examined in experiments combining EGFR-CD533 expression and MEK1/2 inhibition (Dudley et al., 1995. Proc. Natl. Acad. Sci. USA, 92, 7686-7689). The 3.9-fold radiation-induced JNK1 activation within 10 min after MAPK inhibition (FIG. 8B) was reduced to a 2.7 (±0.1)-fold increase upon the expression of dominant negative EGFRCD533 (FIG. 8C). While this represents a 45% reduction of JNK1 activation, the residual 2.7-fold activation still constitutes a significantly higher (P<0.01) activity than the radiation-induced 1.9-fold stimulation of JNK1 in the absence of EGFR-CD533 expression (FIG. 8C).

These results suggest that signaling components downstream of MEK1/2 exerted a negative regulatory effect on the immediate JNK1 activation by radiation. Furthermore, inhibition of radiation-induced EGFR activation combined with MEK1/2 inhibition resulted in reduced JNK-1 activation (FIG. 8C) as compared to MEK1/2 inhibition alone (FIG. 8B). These results suggest that radiation-induced EGFR activation did contribute to the enhanced stimulation of JNK1 under conditions of MEK1/2 inhibition. This occurred despite our finding that radiation-induced EGFR signaling was not required for the immediate activation of JNK1 as shown in FIG. 8C. Importantly, the results demonstrate cross-communication between the MAPK and JNK1 pathways.

The role of Ras in radiation-induced activation of MAPK and JNK1. Having demonstrated the interactions between radiation, EGFR function, and two major downstream signaling cascades, it was important to determine at which level communication between the MAPK and JNK1 pathways occurred. There is considerable evidence that growth factor receptors stimulate both MAPK and JNK1 activities via Ras (Kawasaki et al., 1996; Rausch and Marshall, 1997; Terada et al., 1997). Ras function can be blocked by inhibiting Ras farnesylation which prevents translocation of molecules to the plasma membrane (Suy et al., 1997). Therefore, the dependence of radiationinduced MAPK and JNK1 activation on Ras function was determined.

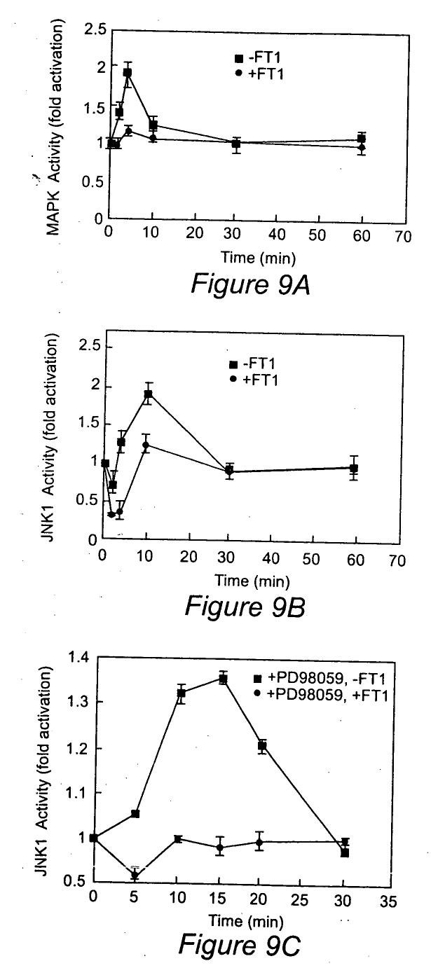

In these experiments, Ras was inhibited by treatment of MDA-TR15-EGFR-CD533 cells with farnesyltransferase inhibitor I (FTI) 24 h prior to irradiation (Manne et al., 1995). This was followed by quantitation of MAPK and JNK1 activities 0-60 min after irradiation. Treatment with FTI inhibited Ras farnesylation and resulted in complete inhibition of radiation-induced MAPK activation (FIG. 9A). This effect was expected since EGF- and radiation-dependent activation of MAPK is known to occur through an EGFR/Ras/Raf-1 signaling pathway (Schmidt-Ullrich et al., 1997; Kavanagh et al., 1998). However, treatment with FTI also completely blocked the ability of radiation to stimulate JNK1 (FIG. 9B), suggesting that JNK1 activation after irradiation occurred via a Ras-dependent pathway.

Additional control experiments were performed to confirm that Ras function played a role in the ability of PD98059 to potentiate JNK1 activation without irradiation. Treatment of MDA-TR15-EGFR-CD533 cells with PD98059 resulted in a transient, 1.35 (±0.02)-fold activation of JNK1 between 5 and 25 min which returned to basal level after 30 min of drug exposure. This response was completely ablated if cells were pre-incubated with FTI for 24 h (FIG. 9C). This finding suggests that cross-communication occurred between the MAPK and JNK1 pathways and identified Ras as one of the components involved in this interaction.

Enhanced c-Jun Ser-63 phosphorylation as a result of MAPK inhibition. In order to examine in more detail the functional consequences of JNK1 activation, we quantified the relative c-Jun Ser-63 phosphorylation in the N-terminal portion of c-Jun in vivo using immunoblotting with antibody specific for Ser-63 phosphorylated c-Jun (Kyriakis et al., 1994). In previous experiments, the activation of JNK1 (FIGS. 7B and 8B-C) was determined by an in vitro immune complex kinase assay using GST-c-Jun (aa 1-139) as substrate.

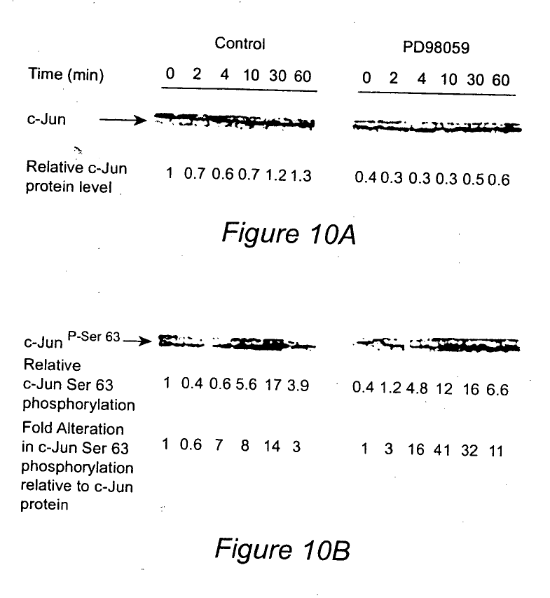

Treatment of cells with PD98059 caused a more than twofold reduction in c-Jun protein levels relative to untreated cells (FIG. 10A). Irradiation alone induced a maximal 14-fold increase of c-Jun phosphorylation at Ser-63 relative to c-Jun protein levels (FIG. 10B), but had no significant effect on steady state c-Jun protein levels over the 60 min examined (FIG. 10A). In contrast, cells irradiated after pretreatment with PD98059 demonstrated a substantial enhancement of relative Ser-63 phosphorylation as quantified in FIG. 10B. This Ser-63 phosphorylation was most pronounced 10 min after irradiation, resulting in a 41-fold increase in c-Jun protein phosphorylation relative to c-Jun protein level. This fivefold greater stimulation of c-Jun phosphorylation under conditions of MEK1/2 inhibition compared to radiation alone was sustained for up to 60 min. The time course of Ser-63 phosphorylation in vivo correlated well with data obtained from the in vitro immune complex kinase assay. The results demonstrate that inhibition of MAPK can redirect signals from EGFR to the JNK1 cascade and that activation of JNK1 leads to enhanced Ser-63 phosphorylation of c-Jun.

The effects of EGFR function, MAPK activation, and JNK-1 signaling on radiation-induced apoptosis. The effects of inhibiting radiation-induced activation of EGFR and MAPK on apoptosis after irradiation were examined in the next set of experiments. Apoptosis was assessed by the Terminal dUTP Nucleotidyl End Labeling assay (Gorczyca et al., 1993) under conditions of EGFR-CD533 expression or MEK1/2 inhibition 24 h after irradiation. Under control conditions, the basal apoptosis rate of MDA-TR15EGFR-CD533 cells was 3.1 (±0.1) % (FIG. 1A). Expression of EGFR-CD533 or MEK1/2 inhibition resulted in an approximate 20% increase in basal apoptosis rates of 3.6 (±0.1) % and 3.7 (±0.1) %, respectively (P<0.05). A single radiation exposure at 2 Gy increased the level of apoptosis to 5.0 (±0.1) % in untreated cells. However, irradiation of cells under conditions of EGFR-CD533 expression or MEK1/2 inhibition, resulted in significantly enhanced rates of 6.4 (±0.1) % and 6.3 (±0.1) %, respectively (P<0.05). These incremental changes in apoptosis reflect a synergistic effect between radiation and disruption of EGFR or MAPK functions (P<0.05). The combination of EGFR-CD533 expression and MEK1/2 inhibition did not enhance the apoptotic response above the effect of either treatment alone (data not shown). These findings suggest that the enhanced rate of apoptosis after EGFR-CD533 expression may be functionally linked to the inhibition of the MAPK pathway.

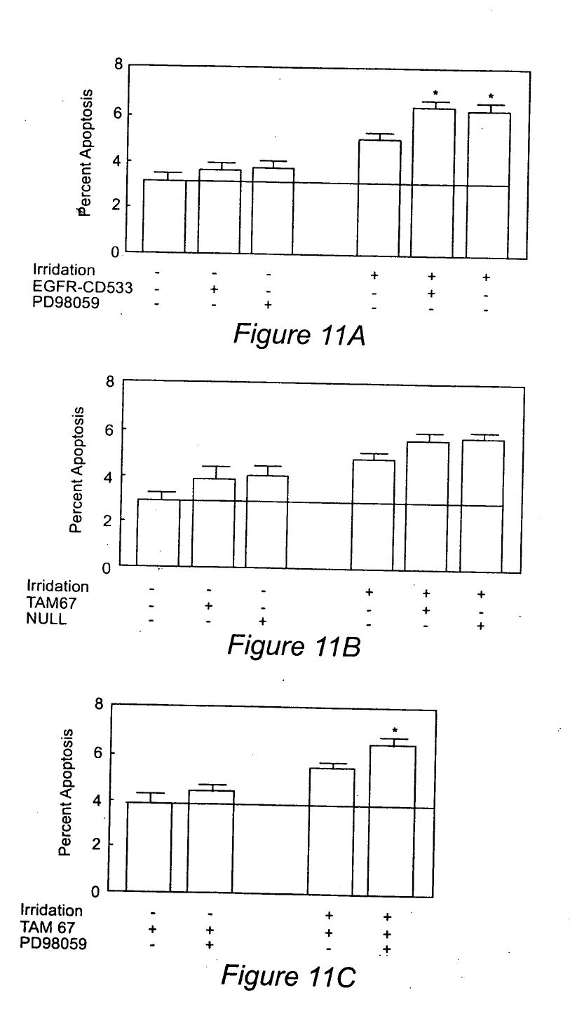

The role of JNK-1 signaling was also assessed with respect to radiation- induced apoptosis. In the absence of a direct inhibitor of JNK-1, the potential role of JNK-1 signaling in radiation-mediated apoptosis was examined through negative modulation of the JNK-1 substrate c-Jun. The dominant negative N-terminally truncated c-Jun, TAM 67, was expressed by infection of cells with a recombinant adenovirus, Ad-TAM 67. In previous experiments with a variety of cell lines, TAM 67 has been shown to inhibit the activation of c-Jun by JNK-1 including AP1-induced transcription (Auer et al., 1998; Janulis et al., 1999; Verheij et al., 1996). Expression of TAM67 in MDA-TR15-EGFR-CD533 cells was determined 48 h after infection with the adenovirus (Ad) by immunoblotting (data not shown). Infection of cells with Ad-null and Ad-TAM67 increased the basal rate of apoptosis to 5.7 (±0.1) % and 5.5 (±0.1) %, respectively (FIG. 11B). Surprisingly, however, irradiation of MDA-TR15-EGFRCD533 cells expressing TAM67 did not significantly blunt radiation-induced apoptosis. These results suggest that the JNK1 substrate, c-Jun, may not be the only determinant controlling radiation-induced apoptosis in mammary carcinoma cells.

In FIGS. 8 and 10 inhibition of MEK1/2 enhanced radiation-induced JNK1 activation was demonstrated. Since radiation-induced apoptosis has also been linked to increased JNK1 activity (Verheij et al., 1996), the question of whether inhibition of MEK1/2 could potentiate radiation-induced apoptosis was next examined. It was surprising to find that expression of TAM67 was unable to modulate the rate of apoptosis after irradiation under conditions of MEK1/2 inhibition (FIG. 11C). Therefore, the effects of MEK1/2 inhibition on JNK1 activation and potentiation of apoptosis in MDATR15-EGFR-CD533 cells can be dissociated.

The importance of EGFR and MAPK functions on cell proliferation after irradiation. An assay for cellular growth potential after irradiation was devised to assess radiation-induced cytotoxicity after repeated radiation exposures of 2 Gy (see Example 1). Cells were irradiated with 2 Gy once-daily for five consecutive days and cell proliferation was measured for seven consecutive days using the NITT method. The effects of radiation-induced EGFR and MAPK signaling were examined by disruption of either function through expression of EGFR-CD533 and inhibition of MEK1/2, respectively.

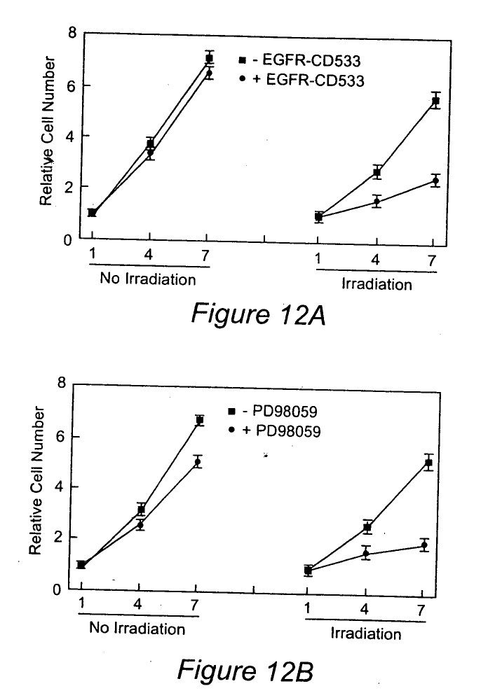

Expression of EGFR-CD533 alone inhibited growth of the MDA-TR15-EGFR-CD533 cells by approximately 10% during 7 days (FIG. 12A, left panel), a modest but significant reduction (P<0.05). However, when cells expressing EGFR-CD533 were irradiated, a 3.1 (±0.1)-fold reduction in cell numbers was observed at 7 days relative to control cells with preserved EGFR function (FIG. 12A, right panel). This data suggests that EGFR protects cells from the cytotoxic effects of repeated radiation exposures and that disruption of EGFR function results in an approximately threefold enhancement of radiation toxicity. Similarly, treatment of MDA-TR15-EGFR-CD533 cells with PD98059 alone resulted in a moderate, but significant retardation of cell growth with a 25% decrease in cell numbers on day 7 (FIG. 12B, left panel) (P<0.05). Irradiation of cells in the presence of the MEK1/2 inhibitor resulted in 5.0 (±0.03)-fold lower cell numbers on day 7 of the growth assay (FIG. 12B, right panel), which represented an even greater enhancement of radiation toxicity than that was induced by the expression of EGFR-CD533 (FIG. 12B, right panel).

This data strongly suggests that EGFR, via MAPK signaling, mediates an important cytoprotective effect during and after exposure to ionizing radiation. Inhibition of this response at the level of EGFR or MAPK essentially ablates the cells' proliferative capacity, thus enhancing radiation toxicity.

### Example 3

### EGFR as Genetic Therapy Target for Carcinoma Cell Radiosensitization

### Example 3

### Materials and Methods

Cell culture. The human MCC line MDA was obtained through the American Type Tissue Collection (ATCC; Rockville, Md.). The MDA-TR15-EGFR-CD533 (MDA-EGFR-CD533) cell line was developed in our laboratory through stable transfection of MDA cells with EGFR-CD533 under expression control of a doxycycline (Dox)-inducible promoter (Contessa et al, 1999). Cells were maintained in RPMI 1640 medium containing 5% tetracycline-free fetal calf serum (RPMI/5FCS) and antibiotics (penicillin/streptomycin) except for exposure 0.5% reduced FCS medium (RPMI/0.5FCS) prior to irradiation as previously described (Reardon et al., 1999; Contessa et al., 1999).

Animals and tumors. Athymic female NCr-nu/nu mice were obtained from the Animal Production Area of the National Cancer Institute-Frederick Cancer Research Facility (Frederick, Md.). Mice used in this study were maintained under pathogen-free conditions in facilities approved by the American Association for Accreditation of Laboratory Animal Care and in accordance with current regulations and standards of the U.S. Department of Agriculture, U.S. Department of Health and Human Services, and the National Institutes of Health. Solitary tumors were produced by subcutaneous inoculation of 10⁷ cells into the right hind leg of 4-6 week old mice from MDA or MDA-EGFR-CD533 tissue culture stocks. All experiments were conducted with tumors 8 to 10 mm in diameter.

Adenoviral transduction. Replication-incompetent Adenovirus (Ad) was produced as described previously (Valerie, 1999; Valerie et al, 2000). The DN EGFR-CD533 cDNA (Kashles et al., 1991), lacking the 533 COOH-terminal amino acids, was kindly provided by A. Ullrich (Max-Planck-Institut fuer Biochemie Martinsried, Germany). Ad-EGFR-CD533 or Ad-LacZ (control vector) expressing the bacterial lacZ reporter gene was produced in 293 cells and purified as described previously (Valerie et al., 2000; Brust et al. 2000). For quantification of transduction efficiency 48 h after transduction with Ad-LacZ, cells expressing β-galactosidase (β-gal) were quantified by counting 500 cells as described (Brust et al. 2000) after counterstaining with safranin O for improved accuracy of identifying LacZ-negative cells (Ho and Lin, 1997). The multiplicity of infection (MOI) was optimized for maximum transduction and minimum cellular toxicity not exceeding 20% in colony formation efficiency.

Immunoprecipitation and immunoblotting assays. Methods for immunoprecipitation of EGFR and Western analysis for EGFR and EGFR-CD533 expression and EGFR Tyr phosphorylation (Tyr-P) were employed as described (Reardon et al, 1999; Contessa et al., 1999). The EGFR Tyr-P levels were assessed in tumor tissue samples excised at certain times after irradiation and frozen in liquid nitrogen. For preparation of tumor lysates frozen tissue was pulverized in liquid nitrogen using mortar and pestle (Dent et al., 1990), tissue was then immediately lysed in ice-cold lysis buffer, 25 mM β-glycerophosphate, pH 7.4, 25 mM Tris, pH 7.4, 10% (v/v) glycerol, 1.5 mM EGTA, 0.5 mM EDTA, 1% Triton X-100, 1 mM Na-orthovanadate, 1 mM Na-pyrophosphate, 15 μg/ml aprotinin, 15 g/ml leupeptin, 2 μM benzamidine, 150 μg/ml PMSF. Tumor tissue lysis was facilitated by repeated passage through 16- to 21 gauge needles. Tissue debris was removed by centrifugation at 14,000-×g for 10 min. Tumor cell lysates were further processed for immunochemical analysis of EGFR and EGFR-CD533 protein expression and EGFR Tyr-P as described above.

Cell treatments and irradiation in vitro. MDA and MDA-EGFR-CD533 cells were seeded in 60 mm dishes at 1.4×10⁵ and cultured for 5 days. On day 3, MDA cells were transduced with Ad-EGFR-CD533 or Ad-LacZ with an MOI of 50, or mock transduced. EGFR-CD533 expression was induced in MDA-EGFR-CD533 cells with Dox at 1 μg/ml for 48 h. For immunochemical analyses, cells were maintained in RPMI/0.5FCS for 16 h prior to EGF treatment or irradiation. Cell processing thereafter followed protocols previously described (Contessa et al., 1999; Valerie et al., 1999 Schmidt-Ullrich et al., 1997). For all radiation experiments a ⁶⁰Co source was used at a dose rate of 1.9 Gy/min.

Immune complex MAP kinase assay. Cell lysates were used for immunoprecipitation with antibody against MAPK (Reardon et al., 1999). Briefly, identical lysate aliquots were incubated with anti-ERK2 (C-14) antibody (Santa Cruz Biotechnology Santa Cruz, Calif.) at 4° C. for 3 h., washed, and immune complexes incubated with MBP for the kinase assays in presence of [³²P]-γ-ATP (5000 cpm/pmol) at 37° C. for 30 min. MBP substrate labeling with ³²P by MAPK was measured following binding to P81 assay paper and liquid scintillation spectroscopy (Reardon et al., 1999).

Colony formation assay. Forty-eight h after Ad transduction or Dox treatment, cells were irradiated with single doses of 1, 2, 3, 4, and 8 Gy and, after incubation for an additional 24 h, harvested and plated for clonogenic survival. The cell numbers plated were adjusted to yield 50-300 colonies/dish for each radiation dose. Cells were cultured for 12 days, fixed and stained with crystal violet, colonies containing ≧50 cells were counted for quantification of the clonogenic surviving fraction and radiosensitization computed by D ratios (Fertil et al., 1984). The survival curves were fitted by use of a linear-quadratic model (Hall, 1994).

Expression of EGFR-CD533 by Dox regulation or Ad-EGFR-CD533 infusion in vivo. EGFR-CD533 expression in MDA-EGFR-CD533 tumor xenografts was induced by Dox by seven intraperitoneal injections (300 μg; 1 μg/μl) over 90 h at approximately 12 h intervals prior to a 4 Gy single radiation exposures. The same twice daily Dox treatment was applied prior to/during the repeated 3×1.5 Gy exposures, except that Dox treatment was initiated 24 h before the first radiation exposure. Dox treatment was continued 20 h beyond the last irradiation to the time of tumor harvest and preparation of single cell suspensions for immunochemical and survival analyses.

Tumor xenografts were infused with Ad-LacZ or Ad-EGFR-CD533 using a special flow-rate controlled, positive pressure infusion device (Brust et al., 2000; Prabhu et al, 1998) with a constant flow rate of approximately 0.5 μl/min. Initial experiments explored improved Ad delivery of 10¹⁰ pfu (in 0.12 ml PBS) through variation of the infusion methods using 4 or 6 tracks during a single 40-min infusion. For the most efficient transduction in vivo, six 30-gauge needles were positioned as two sets of 3 needles in opposing directions penetrating 60% of the tumor diameter. Similar spacing between the needles aimed at the widest distribution of Ad within the tumor, and each needle was retracted 1 mm every 10 min during a 40 min infusion. The 2×6-track method was performed as 6-track infusion on two consecutive days with the second infusion positioning of two sets of 3 needles at right angles to the initial 6-track infusion. A Bee Hive Controller and a Baby Bee Syringe Pump (Bioanalytical Systems, Inc. West Lafayette, Ind.) were used for all viral infusions. All infusions were performed on fully anaesthetized mice.

Irradiation of tumor xenografts. The radiosensitizing effects of EGFR-CD533 in vivo were tested in MDA-EGFR-CD533 tumors after Dox treatment of mice or in MDA xenografts after infusion of Ad-EGFR-CD533. Repeated 3×1.5 Gy radiation exposures in vivo were scheduled relative to twice daily Dox administrations as described above. Whole body irradiation at 1.5 Gy/day for 3 consecutive days was performed in a plastic container assuring exposure of each mouse to full dose. Tumors in control mice were infused with Ad-LacZ or Ad-EGFR-CD533 and subjected to mock irradiation. For immunohistochemical analysis of radiation-induced EGFR activation, as assessed by Tyr-P levels, mice were exposed to a single whole body dose of 4 Gy followed by tumor excision at specified times and immediate freezing of tumor tissue in liquid nitrogen.

Ex vivo colony formation assay. The in vivo radiation toxicity was quantified using an ex vivo clonogenic survival assay (Brown and Lemmon, 1990) since growth delay assays were complicated by the retarded growth of MDA-CD533 tumors in vivo. Mice were sacrificed 24 h after irradiation, tumors excised and single cell suspensions were generated in 1.5 ml of collagenase (280 U/ml) and 1.5 ml of a DNase (31 U/ml), pronase (225 U/ml), and collagenase (3280 U/ml) in 15 ml of RPMI/5FCS and antibiotics. After 2-3 h of digestion, single cells were passed through a 40 μm mesh filter, washed twice and counted. The cells were then plated for colony formation assay as described above. The data obtained from an individual tumor represents the average value of clonogenic assay performed in quadruplicate.

Statistical analysis. All data are means±standard deviation (S.D.), unless 10 otherwise specified. Statistical comparisons between clonogenic survival curves were carried out by use of the F-test. The Student's t test was applied for all other statistical evaluations. A P<0.05 was considered to be statistically significant. All P values reported are two-sided. All statistical analyses were carried out with the SAS software package (version 8.0; SAS Institute, Inc., Cary, N.C.). The dose enhancement ratio (DER) was derived from the D 1 5 ratio in single dose clonogenic survival assays and the fold difference in ex vivo clonogenic survival after repeated radiation exposures in vivo.

### Example 3

### Results

Transduction of MIDA Cells with Ad-EGFR-CD533 Blocks Radiation-Induced Activation of EGFR and MAPK. MDA cells were selected because they express intermediate levels of EGFR and are autocrine growth regulated by this receptor that facilitates a proliferation response at low EGF concentrations and after radiation exposures in the therapeutic dose range between 1 and 5 Gy. This proliferation response is mediated by MAPK (Schmidt-Ullrich, 1997; Reardon et al., 1999). We have also characterized the MDA-EGFR-CD533 cell line including its role of EGFR-CD533 in modulating cellular radiation response (Contessa et al. 1999).

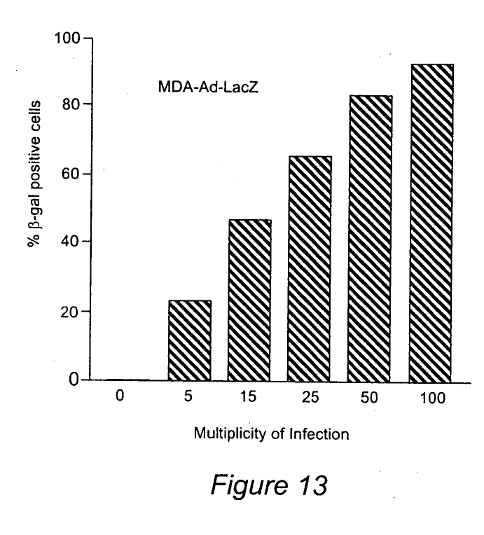

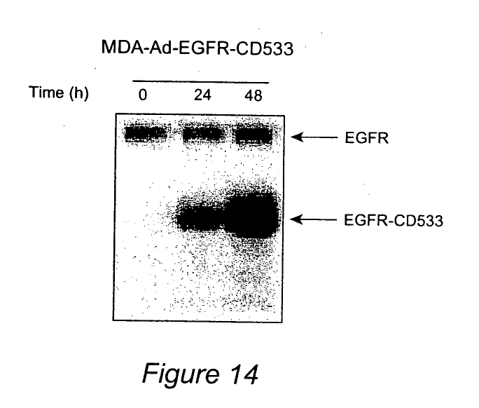

The exposure of 60-80% confluent MDA cells to Ad-LacZ at an MOI of 50 yielded>80% transduction efficiency using β-galactosidase staining 48 h after transduction as an endpoint (FIG. 13). The EGFR-CD533 expression kinetics demonstrated detectable expression 24 h after Ad-EGFR-CD533 transduction, and approached maximum values by 48 h (FIG. 14).

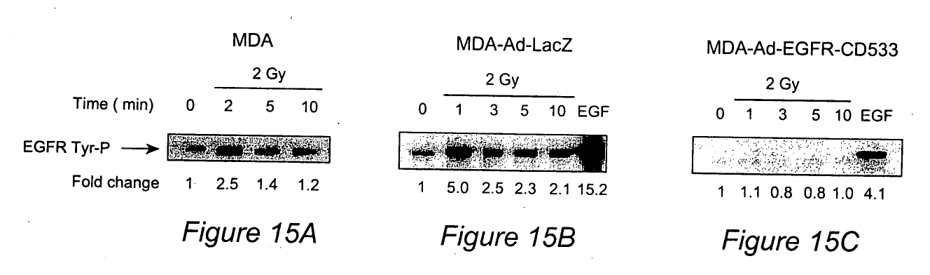

MDA cells demonstrated a 2.5-fold activation of EGFR as reflected in increased Tyr-P (FIG. 15A). This EGFR activation profile was preserved, when MDA cells were transduced with Ad-LacZ (FIG. 15B). However, when EGFR-CD533 was over-expressed the radiation-induced activation of EGFR was completely ablated. The minimum residual Tyr-P after EGF treatment reflects >90% inhibition upon expression of EGFR-CD533 (FIG. 15C).

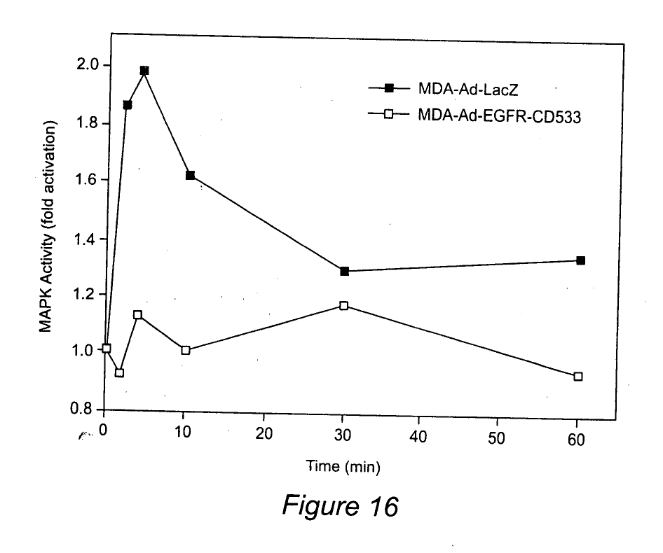

In previous work it has been demonstrated that MAPK is an important downstream target of EGFR after radiation exposure of cells and that its activation correlated with a cellular proliferative response (Schmidt-Ullrich, 1997; Kavanagh et al., 1995). We confirm here that upon transduction of MDA cells with Ad-EGFR-CD533, the MAPK response was completely ablated (FIG. 16), demonstrating that EGFR-CD533 expressed from an Ad responded indistinguishable from MDA-CD533 cells representing a DN EGFR phenotype (Reardon et al., 1999).

These results demonstrate that almost identical results are seen with EGFR-CD533 expressed from an Ad as those seen with stably transfected MDA-EGFR-CD533 cells (Reardon et al., 1999).

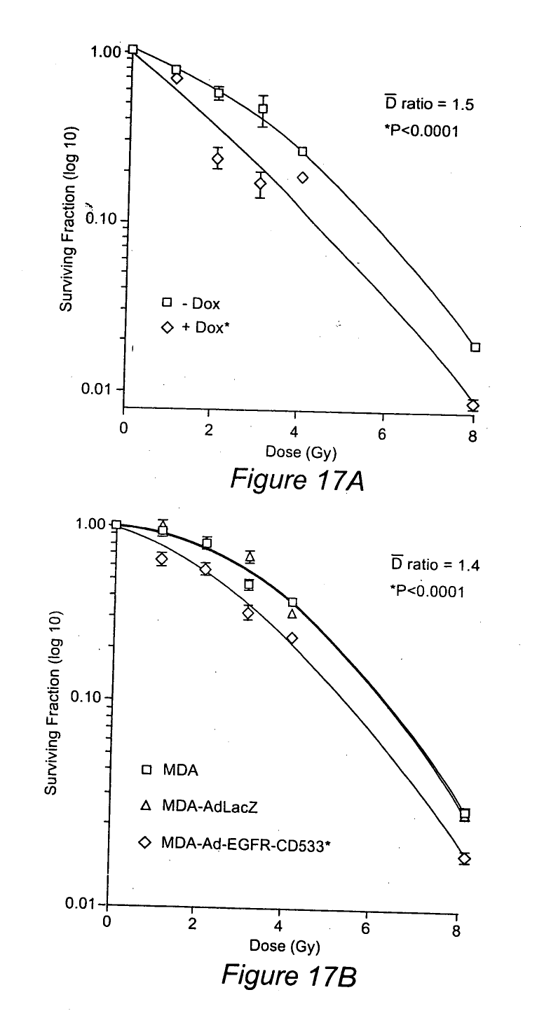

EGFR-CD533 Expression Radiosensitizes Mammary Carcinoma Cells in vitro. Having demonstrated that the expression of DN EGFR-CD533 disrupted radiation-induced ECFR activation, the question of whether the expression of EGFR-CD533 resulted in radiosensitization of MDA cells was examined. In single radiation dose response experiments, MDA-EGFR-CD533 cells (±Dox) were exposed to radiation doses between 1-8 Gy followed by clonogenic survival 24 h later (Hahn and Little, 1972). The MDA-EGFR-CD533 cells, upon induction of EGFR-CD533, exhibited a significant (P<0.0001) radiosensitization with a DER of 1.5 (FIG. 17A). These results were compared with untransduced, control transduced (Ad-LacZ) and Ad-EGFR-CD533 transduced MDA cells. The radiation survival curves for untreated and Ad-LacZ transduced MDA cells were similar to that of MDA-EGFR-CD533 cells without expression of EGFR-CD533 (−Dox). In contrast, MDA cells transduced with Ad-EGFR-CD533 demonstrated significant (P<0.0001) radiosensitization, similar to that observed for MDA-EGFR-CD533 cells under conditions of EGFR-CD533 over-expression (+Dox), yielding a DER of 1.4 (FIG. 17B).

This data demonstrates that over-expression of EGFR-CD533 results in direct radiosensitization of MDA cells after single radiation exposures in addition to inhibition of the previously described radiation-induced proliferation response that is most impressively shown after repeated radiation exposures (Contessa et al., 1999).

Radiation-induced Activation of EGFR and EGFR-CD533 Expression in vivo. In order to correlate in vivo radiation responses of tumors with the expression of EGFR-CD533, the conditions for maximum EGFR-CD533 expression in xenografts from MDA-EGFR-CD533 cells were employed using repeated intra-peritoneal Dox administrations. By comparison, we quantified the expression of EGFR-CD533 after intratumoral injection of Ad-EGFR-CD533 in vivo.

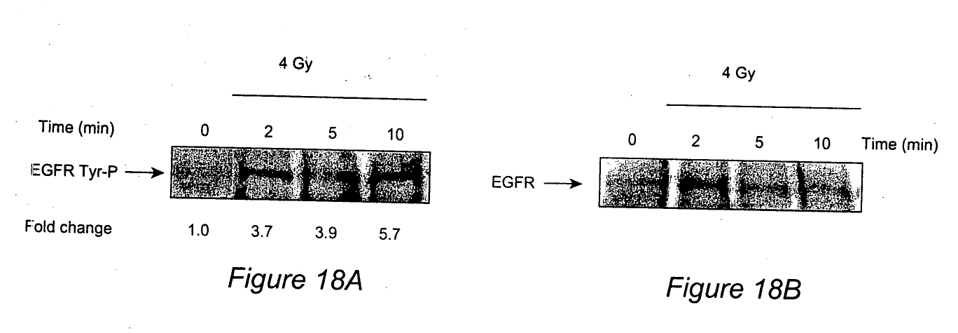

Exploratory radiation dose analyses in vivo using an MDA xenograft model demonstrated that single 4 Gy exposures of tumors yielded maximum radiation-induced EGFR activation as reflected in 2.5- to 5-fold increases in Tyr-P within 2 to 10 min (FIG. 18A). In addition we determined that the interactions between radiation-induced proliferation responses and EGFR function were best quantified using 3×1.5 Gy daily radiation exposures which resulted in a 50% ex vivo clonogenic survival relative to un-irradiated control tumors (data not shown).

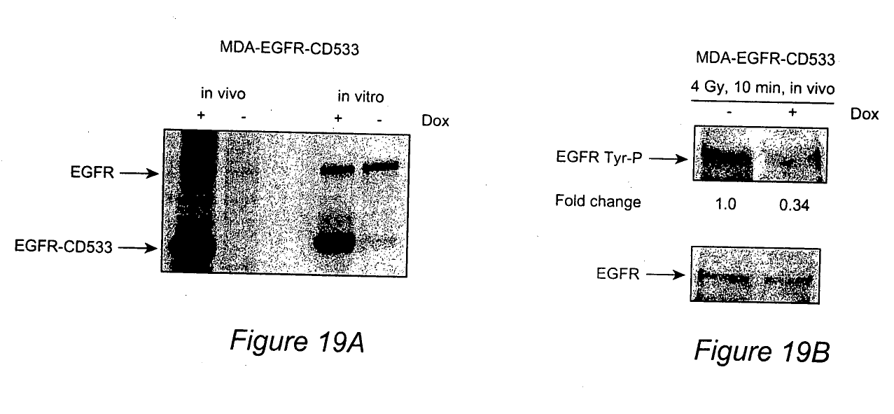

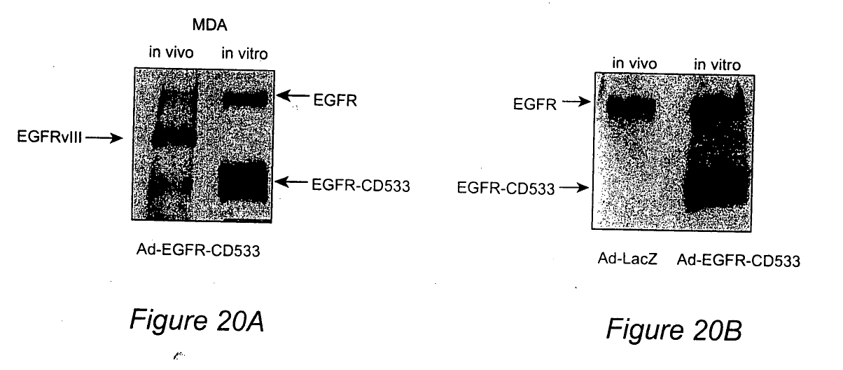

FIG. 19A demonstrates that Dox treatment resulted in similar degrees of EGFR-CD533 over-expression in vitro and in vivo. Importantly, Dox induced expression of EGFR-CD533 in vivo resulted in a 3-fold reduction in radiation-induced activation of EGFR, as assessed by Tyr-P, within 10 min of a single 4 Gy exposure in MDA-CD533 xenografts (FIG. 19B). The injection of Ad-EGFR-CD533 into MDA tumors resulted in EGFR-CD533 expression similar to that of endogenous EGFR (FIG. 20A). Based on total cellular protein, the expression levels of EGFR and EGFR-CD533 appear lower than in vitro, an observation that warrants additional investigation. The finding is further corroborated by the result that cells transduced in vivo with Ad-LacZ or Ad-EGFR-CD533 appear to express higher levels of the wild-type and DN receptor after 24-96 h maintenance in vitro (FIG. 20B). In addition, an EGFRvIII band was found in MDA tumor cell lysates (FIG. 20A), as verified by use of an anti-EGFRvIII mAb (data not shown).

Optimization of Intratumoral Infusion of Ad-EGFR-CD533 into MDA Xenografts

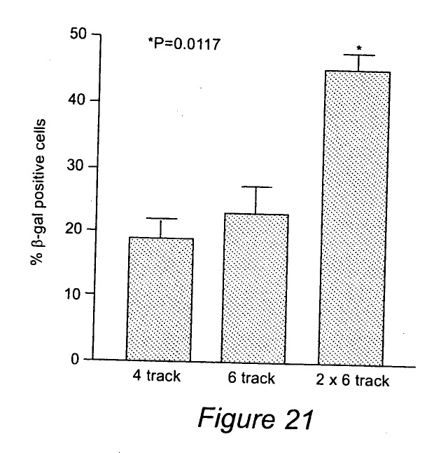

To establish optimal infusion conditions of Ad into MDA xenografts a fixed dose of Ad (10¹⁰ pfu) was infused by different administration methods (FIG. 21). The average transduction rates with Ad-LacZ, using single 4- or 6-track infusions were 18 or 24%, respectively, quantified on single cells liberated after digestion of infused tumors. The most significant (P=0.0117) gain was achieved by 6-track infusions on two consecutive days with manual needle retraction, at 10 min intervals yielding transduction rates >40%.

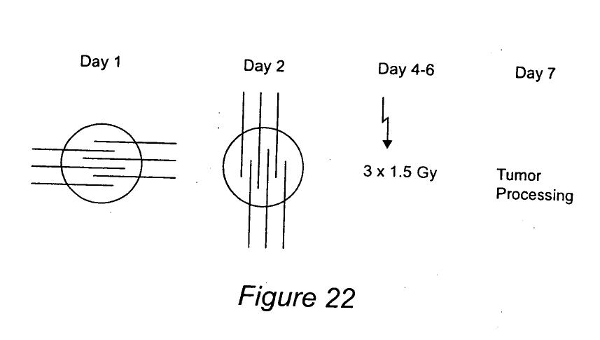

EGFR-CD533 Expression in vivo Results in Tumor Radiosensitization. In experiments described above it has been demonstrated that the radiosensitization under condition of EGFR-CD533 over-expression is independent of the method of EGFR-CD533 induction. Consecutive experiments aimed at verifying the in vitro effects of EGFR-CD533 expression on cellular radiation responses in vivo. These radiosensitizing effects of EGFR-CD533 were tested using an MDA xenograft model in nude mice in whom we had defined the optimal radiation schedule for the relatively radiosensitive MDA xenograft tumors, a schematic representation of which is given in FIG. 22.

As experimental reference model we used tumors from MDA-EGFR-CD533 cells (Reardon et al., 1999) because all tumor cells could be expected to express EGFR-CD533 at maximum levels after intraperitoneal Dox administration. Under these conditions the kinetics of maximum EGFR-CD533 expression were identical to those described in vitro (Contessa et al, 1999). Thus these conditions could be expected to yield greatest radiosensitization. Of all tumors tested, >80% expressed expected high levels of EGFR-CD533, but <20% expressed low or minimal levels of the transgene. Only tumors expressing high levels of EGFR-CD533 were used for the radiosensitization experiments.

Since EGFR-CD533 expressing tumors demonstrated an approximately 20% reduction in clonogenicity (data not shown), comparisons were limited to radiation responses of cells with or without EGFR-CD533 expression. Normalized clonogenic survival data, shown in FIG. 4C, were generated and demonstrated a significant (P<0.0001) 38% reduction in clonogenic survival, corresponding to a DER of 1.6.

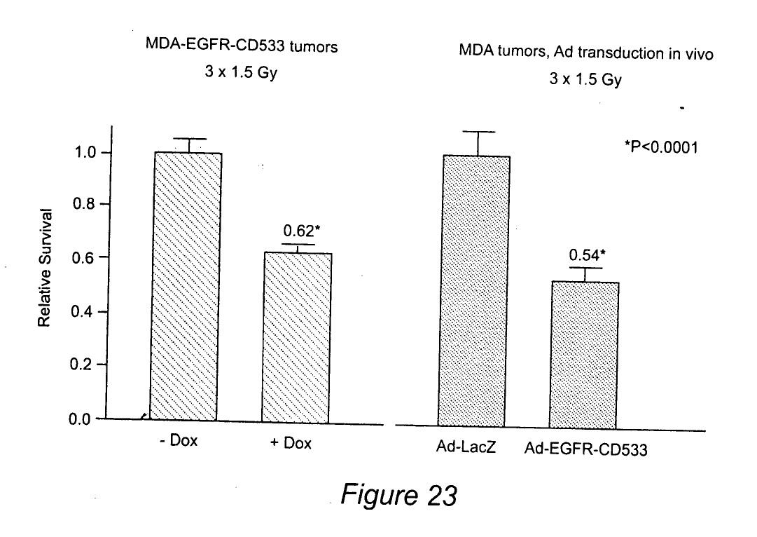

As an additional step towards therapeutic application we pursued the cancer genetic therapy approach of delivering the EGFR-CD533 as an Ad vector. For these experiments optimal conditions of Ad delivery were employed. Under the conditions of >40% transduction of MDA cells with EGFR-CD533 a significant (P<0.0001) 46% increase in radiation toxicity was observed relative to Ad-LacZ vector controls, amounting to a DER of 1.85 (FIG. 23).

Thus these data demonstrate that the expression EGFR-CD533 provides significant radiosensitization of mammary carcinoma cells in vitro and in vivo using relatively modest radiation doses.

### Example 4

### Radiosensitization of Malignant Glioma Cells through Over-Expression of Dominant-Negative EGFR

### Example 4

### Materials and Methods

Cell Lines and Reagents. Two human glioma cell lines were used in the present study; U-373 MG, originally isolated from a human anaplastic astrocytoma, and U-87 MG, a human glioblastoma cell line, were obtained through the American Type Tissue Collection (ATCC; Rockville, Md.). U-87 MG cells express wild-type p53 (p53wt), whereas U-373 MG cells express a mutated p53 (p53mut) gene product (Russell et al., 1995; Chen et al. 1996). Human epidermoid carcinoma A 431 cells were also obtained from ATCC. The cell lines were tested for mycoplasma contamination monthly and only negative cells were used for experimentation.

Protease and phosphatase inhibitors and other chemical reagents were purchased from Sigma Chemical Co. (St.Louis, Mo.). All electrophoresis reagents were from BioRad (Hercules, Calif.). The Minimum Essential Medium γ Medium (MEM Alpha) and the Penicillin-Streptomycin antibiotics were from GIBCO-BRL (Rockville, Md.), and fetal bovine serum (FBS) was from Intergen (Purchase, N.Y.).

The following immunological reagents from Neo Markers (Fremont, Calif.) were used: the monoclonal antibody (mAb) cocktail (Ab14) reacting with both C- and N-terminal domains of EGFR, the anti-ErbB2 mAb cocktail (Ab10), reacting with both the extracellular and cytoplasmic domains of ErbB2, and the anti-ErbB3 mAb (Ab7) and anti-ErbB4 mAb (Ab4), that react with the cytoplasmic domains of ErbB3 and ErbB4, respectively. Other antibodies were: anti-EGFR mAb from Transduction Laboratories (PharMingen/Transduction Laboratories, Los Angeles, Calif.), the anti-EGFRvIII mAb reacting with the N-terminal truncation of EGFR, DH8.3 (AbCam Limited, Cambridge, UK), the immunoprecipitating anti-EGFR mAb, Ab5, the antiphospho-Tyr mAb, Ab2, and peroxidase-conjugated goat anti-mouse Ab (Oncogene Science, Cambridge, Mass.). Protein A agarose was from BioRad (Hercules, Calif.).

Mice and Tumors. Athymic female NCr-nu/nu nude mice were obtained from the National Cancer Institute-Frederick Cancer Research Facility (Frederick, Md.). Animals were maintained under specific-pathogen-free conditions in facilities approved by the American Association for Accreditation of Laboratory Animal Care and in accordance with current regulations and standards of the U.S. Department of Agriculture, U.S. Department of Health and Human Services, and the National Institutes of Health. Solitary tumors were produced by subcutaneous inoculation of 10⁷ Cells into the right hind leg of 2 to 3 month old mice. Tumor cell suspensions were prepared from U-87 MG cells grown as monolayers in vitro. Experiments were initiated when the tumors had reached a size of 8-10 mm in diameter.

Recombinant Adenoviruses and in vitro Transduction Conditions. Replication incompetent Ad was produced in bacteria as previously described (Valerie. 1999; Valerie et al., 2000). The mutant EGFRCD533 cDNA (Kashles et al., 1991), lacking the 533 carboxyl-terminal amino acids, was kindly provided by A. Ullrich (Max-Planck-Institut fuer Biochemie Martinsried, Germany). An Ad expressing the bacterial lacZ reporter gene (AdLacZ) was used as control virus (Valerie et al., 2000; Brust et al. 2000). The transduction rate in AdLacZ transduced cells expressing β-galactosidase (β-gal) was determined by x-gal staining after 5-bromo-4-chloro-3-indolyl-β-D-galactopyranoside incubation for 24 h as previously described (Brust et al. 2000). To identify lacZ gene-negative cells accurately, safranin O was used for counterstaining (Ho et al. 1997). Ad-EGFR-CD533 or AdLacZ was produced in 293 cells as described and purified by double CsCl gradient centrifugation followed by dialysis against 13% glycerol in phosphate-buffered saline (Valerie et al., 2000; Brust et al. 2000). Virus was frozen in liquid nitrogen and stored at −70° C. until further use. Titration by standard plaque assay indicated typical titers of 2×10¹¹ plaque-forming units per ml (pfu/ml).

Transduction of the cell lines with Ad-EGFR-CD533 or AdL acZ was carried out by diluting viral stocks with PBS, adding adenoviral preparations to cell monolayers (60-mm dish) and placing dishes on a rocker with gentle continuous agitation in a tissue culture incubator for 4 h at 37° C. followed by medium change. The multiplicity of infection (MOI) (pfu/cell) was optimized for maximum transduction and minimum cell toxicity.

Cell Treatments and in vitro Irradiation. U-87 MG and U-373 MG cells were seeded at 1.9×10⁵ (60-mm-dish)/8.0×10⁵ (100-mm dish) or 1.5×10⁵ (60 mm-dish)/4×10⁵ (100-mm dish), respectively, and cultured for a total of 4 to 5 days in MEM Alpha containing 10% FBS (MEM Alpha/1OFBS) and penicillin/streptomycin. For clonogenic survival analyses, cells were transduced on day 3 with Ad-EGFR-CD533, AdLacZ or left untransduced in 1 ml of medium (60-min dish). For immunochemical analyses after irradiation or EGF treatments, media was replaced by low serum MEM Alpha/0.5FBS for 16 h prior to irradiation or EGF treatment. In all radiation experiments, cells were irradiated at a dose rate of 1.8 Gy /min using a ⁶⁰CO source. Cells were maintained at 37° C. throughout the experiment except for irradiation at 20° C. For time course experiments, cells were irradiated with a single dose of 2 Gy and incubated for the times specified. Thereafter, medium was removed and cells were washed once in ice-cold PBS, immediately frozen on dry ice, and stored at −70° C. until further processing.

EGFR Tyr-P and Western blot analysis. Immunoprecipitations and Western blotting were performed as described (Schmidt-Ullrich et al. 1996); briefly, frozen cells were lysed in ice-cold lysis buffer. Cell lysis was facilitated by repeated passage through a 20-gauge needle and insoluble material was removed by centrifugation at 14,000×g for 10 min. For time course experiments, equivalent amounts of protein, quantified by the Bradford Protein Standard Assay (BioRad, Hercules, Calif.) were reacted with Ab5 at 4° C. for 90 min followed by incubation with protein A-agarose for 45 min. Agarose was recovered by centrifugation and immunoprecipitates were washed once in lysis buffer and twice in PBS. Proteins were size fractionated in 6% SDS-polyacrylamide gels, transferred electrophorectically onto nitrocellulose membranes (BioRad, Hercules, Calif.), and probed with anti-phospho-Tyr mAb Ab2 (EGFR Tyr-P studies) or anti-EGFR mAb (EGFRprotein studies) followed by incubation with peroxidase-conjugated secondary Ab. After developing with CDP-Star (Tropix, Inc., Bedford, NIA), autoradiograms were quantified using Sigma Scan software (Jandel Scientific, San Rafael, Calif.) (Schmidt-Ullrich et al. 1996). Ab14 was used for Western blotting of EGFR, Ab 10 for ErbB2, Ab7 and Ab4 for ErbB3 and ErbB4, respectively.

Colony Formation Assay. Cells were incubated on day 3 with either Ad-EGFR-CD533, AdLacZ or left un-transduced in 1 ml of medium (60-mm-dish) for 4 h followed by medium change. After 48 h, cells were irradiated with single doses of 2, 4, and 8 Gy and, after incubation for an additional 24 h, harvested and plated for clonogenic survival. For repeated radiation exposure experiments, cells were exposed once daily to 2 Gy for 3 consecutive days without medium change. The number of cells was adjusted to generate 50-300 colonies per dish at each radiation dose and plated into 4-well 6 cm culture dishes. Cells were incubated at 37° C. with 5% CO₂ for 12-14 days, stained with crystal violet, and colonies containing ≧50 cells were counted to determine surviving fraction (Hall, 1994).

Adenoviral Infusion in U-87 MG Tumors and In Vivo Irradiation. At tumor diameters of 8 to 10 mm., AdLacZ (control) or Ad-EGFR-CD533 was delivered intratumorally as follows. Six 30-gauge needles were positioned as two sets of 3 needles in a triangular arrangement in opposing directions and penetrated to approximately 60% of the diameter of the tumor with appropriate spacing between the needles to allow for maximal Ad distribution. In addition, each needle was retracted 1 mm every 10 min during the infusion. A Bee Hive Controller and a Baby Bee Syringe Pump (Bioanalytical Systems, Inc. West Lafayette, Ind.) were used for all Ad infusions. All infusions were performed on fully anaesthetized mice with a total volume of 20 μl per needle containing 1×10⁹ pfu AdLacZ or Ad-EGFR-CD533 using a PBS vehicle. The flow rate was 0.5 μl /min. At 72 h post-infusion, mice were immobilized in a plastic box, and animals were centered in a 18×18-cm field. A single 3 Gy dose of gamma radiation was delivered whole body on 3 consecutive days at a dose rate of 2 Gy/min using a ⁶⁰Co source. Control tumors infused with AdLacZ or Ad-EGFR-CD533 were handled under mock radiation conditions.

Measures of Tumor Radiosensitization. An ex vivo colony formation assay was performed to assess radiosensitization in vivo. Twenty-four h after irradiation, mice were sacrificed and tumors were excised and digested to single cell suspension in 1.5 ml of collagenase (280 U/ml) and 15 ml of an enzyme cocktail [DNase (30.58 U/ml), pronase (225 U/ml), collagenase (3280 U/ml)], contained in 15 ml of MEM Alpha/10FBS plus antibiotics. After 2 to 3 h of digestion, single cells were filtered through a 40-micron mesh filter, washed twice with media and counted. The cells were then plated for colony formation assay as described above.

Statistical analysis. The Student's t test was applied to establish statistical significance.

### Example 4

### Results

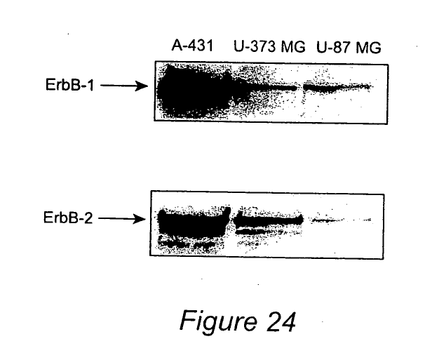

Characterization of EGFR- and ErbB2-protein expression. In order to define the expression levels of EGFR and ErbB2 in the two glioma cell lines, surface receptor expression for each of the two cell lines was confirmed relative to the A-431 cells using mAbs for EGFR and ErbB2. Immunoblotting experiments with Ab14 against EGFR revealed a single 170 kD band in A-431, U-373 MG. and U-87 MG cell lysates (FIG. 24) However, the EGFR expression levels in the two glioma cell lines were substantially lower than in A-431 cells. No additional 145-155 kD band, representing the receptor with a truncated COOH-terminal domain (EGFRvIII) was evident. Western blotting with Ab 10 showed similar ErbB2 protein levels of the 185 kD protein in A-431 and U-373 MG cells, whereas the ErbB2 expression level in U-87 MG cells was lower (FIG. 24). No ErbB3 or ErbB4 and no EGFRvIII bands were found in U-87 MG and U-373 MG cell lysates after immunoblotting with Ab7, Ab4, and anti-EGFRvIII respectively (data not shown). The results demonstrate that both glioma cell lines express significant levels of EGFR and ErbB2.

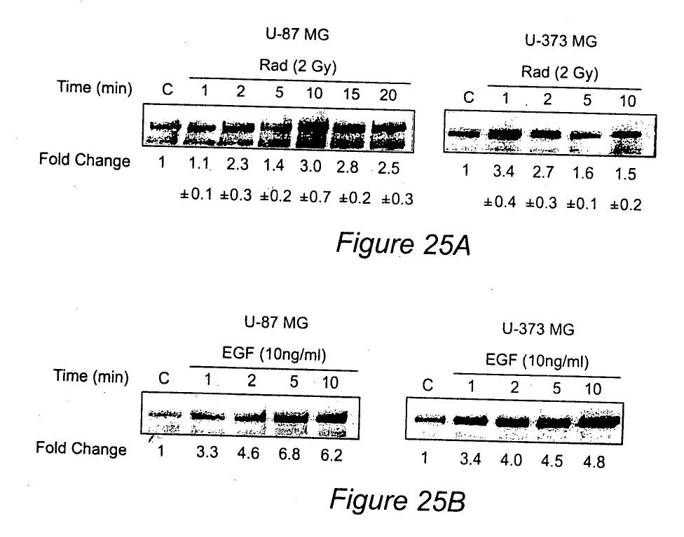

Radiation-induced activation of EGFR Tyr-P in U-87 MG and U-373 MG cell lines. It has been previously demonstrated that radiation in the dose range of 0.5-5 Gy stimulates EGFR Tyr-P in MCF 7, MDA-MB-231, and A-431 cells (Schmidt-Ullrich et al. 1997). In order to test these responses in the malignant glioma cells, U-87 MG and U-373 MG, EGFR, activation after radiation and EGF exposure was quantified by relative levels of EGFR Tyr-P using Western analysis (FIG. 25). A 2 Gy radiation exposure resulted in maximum stimulation of EGFR activation in U-373 MG cells within 1 min, whereas U-87 MG cells showed a prolonged response with a maximum EGFR activation at 10 min (FIG. 25A). In three independent experiments, means of 3.0 (±0.7)-(U-87 MG; 95% confidence interval: 1.86, 3.18; P<0.005) to 3.4 (±0.4)-(U-373 MG; 95% confidence interval: 1.84, 3.28; P<0.001) fold increase relative to the controls were observed; responses in line with those previously found for human carcinoma cells (Schmidt-Ullrich et al. 1997). EGF exposure led to a prolonged increase of EGFR Tyr-P in both cell lines (FIG. 25B).

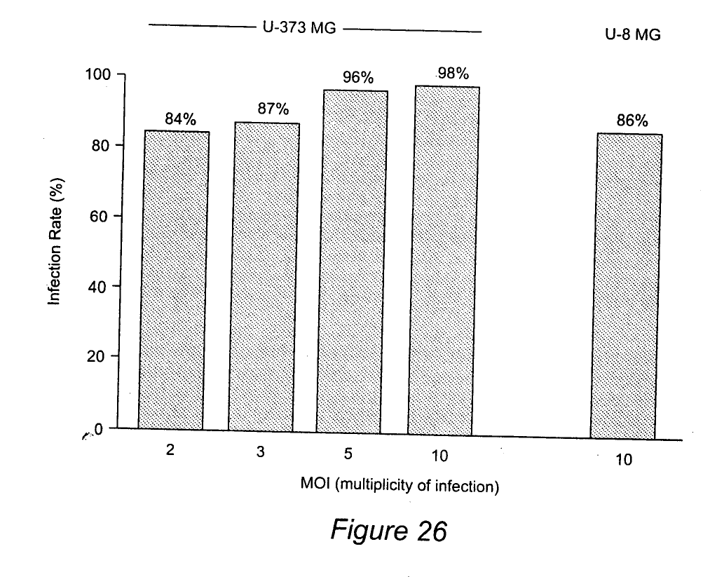

Transduction of U-87 MG and U-373 MG malignant glioma cells in vitro. The overexpression of EGFR-CD533 was studied through the genetic approach of transducing cells with Ad-EGFR-CD533. To optimize the MOI for maximum transduction with minimum cell toxicity in U-87 MG and U-373 MG cells, we evaluated different MOI's between 3 and 30 for the transduction of cells with AdLacZ and Ad-EGFR-CD533 were examined. 10 MOI for U-87 MG and 3 MOI for U-373 MG cells produced 85-90% transduction efficiencies (FIG. 26). Under these conditions, the survival rates by colony formation were 96% and 80% for U-87 MG and U373 MG cells, respectively, relative to untransduced controls (data not shown). Overall, U-373 MG cells demonstrated a greater sensitivity to adenoviral transduction than U-87 MG cells (FIG. 26). The expression of EGFR-CD533 was detectable 24 h after transduction, and approached maximum values by 48 h, as previously described (see Example 2).

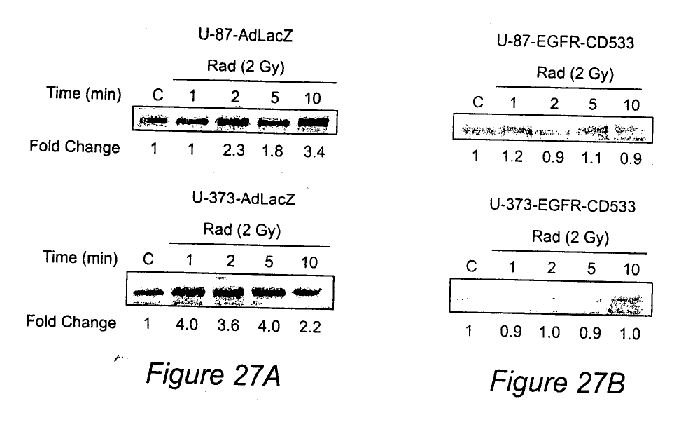

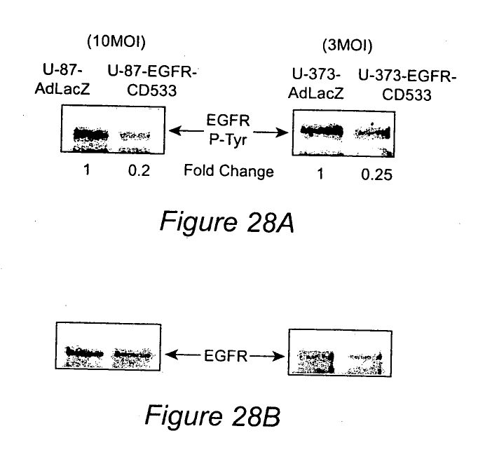

Inhibition of radiation-induced activation of EGFR Tyr-P by over-expression of EGFR-CD533 via Ad-mediated gene transfer. Previous studies have established that expression of EGFR-CD533 does not affect the expression levels of EGFR, but rather disrupts EGFR function and Tyr-P autophosphorylation. (Example 2). Here, the effect of EGFR-CD533 expression on radiation-induced activation of EGFR in U-87 MG and U-373 MG glioma cells was investigated. The studies were carried out using Ad-EGFR-CD533 to over-express EGFR-CD533, and an AdLacZ control virus was used to assess to the effects of vector alone on EGFR Tyr-P. The results shown in FIG. 27. The results shown in FIG. 27A demonstrate that AdLacZ did not affect the radiation-induced activation of EGFR in U-87 MG and U-373 MG cells. However, as shown in FIG. 27B, EGFR activation was completely inhibited in U-87 MG and U-373 MG cells under conditions of EGFR-CD533 over-expression. After transduction with Ad-EGFRCD533, the basal Tyr-phosphorylation levels of EGFR were reduced to 12-20% and 75-96% (range in three independent experiments) in U-87 MG and U-373 MG cells, respectively, relative to AdLacZ controls (FIG. 28A), without effecting basal EGFR-protein levels (FIG. 28B).

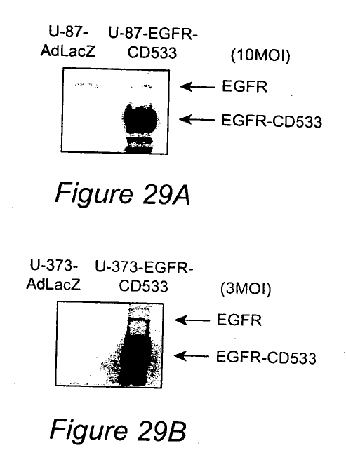

FIG. 29A-B illustrates the expression levels of EGFR-CD533 compared to EGFR at the time of radiation exposure 48 hours after transduction. The expression level of the newly expressed EGFR-CD533 was substantially higher than the expression of the endogenous wild-type EGFR. This data supports previously published data on stably transfected MDA-MB-231 mammary carcinoma cells (see Example 2), demonstrating that the expression of EGFRCD533 almost completely inhibits EGFR function.

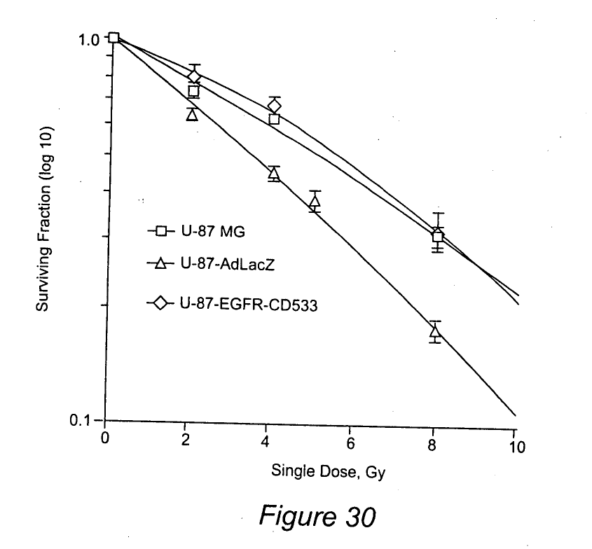

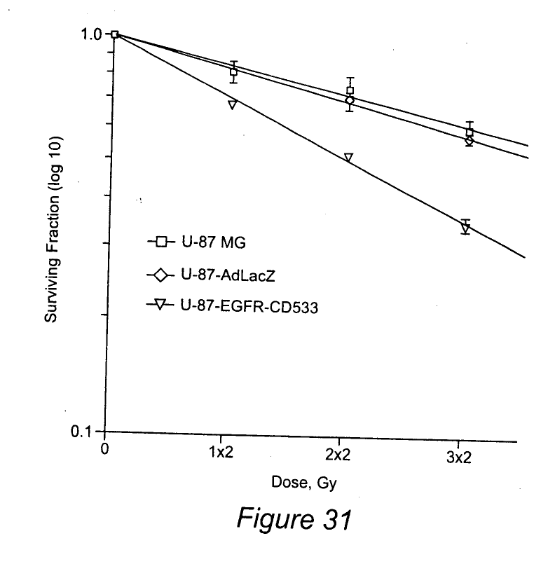

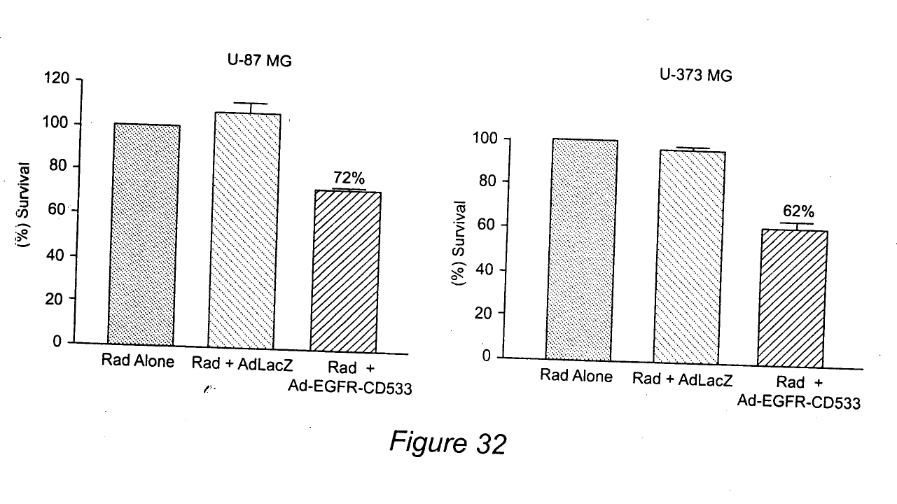

In vitro radiosensitivity of U-87 MG and U-373 MG cells under conditions of EGFRCD533 expression. To assess the effect of inhibition of EGFR function through over-expression of EGFR-CD533 on the radiosensitivity of malignant glioma cells, the single dose response colony formation assay was carried out. As shown in FIG. 30, U-87 MG cells transduced with AdEGFR-CD533 were more radiosensitive over a range of radiation doses compared to the AdLacZ control or untransduced cells. The dose for a survival of 37% (D37) in the U-87-EGFR-CD533 cells was 1.4- to 1.5-fold lower relative to transduction with AdLacZ or untransduced U-87 MG cells. This effect was amplified in repeated radiation exposure experiments, 3×2 Gy, yielding a D37 ratio of 1.8 to 2.0 in comparison to controls (FIG. 31). This reduced clonogenic survival for cells expressing EGFR-CD533 was similar in the U-373 MG cells with a 38% survival reduction after a single dose of 4 Gy (P<0.0001; FIG. 32).

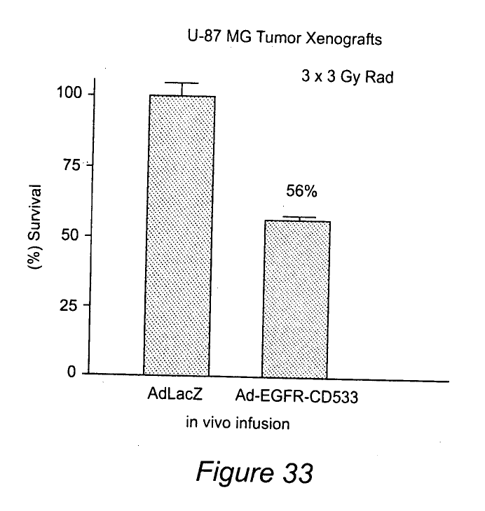

In vivo radiosensitization in U-87 MG tumor xenografts after Ad-EGFR-CD533 infusion. To determine the effect of EGFR-CD533 on tumor radiosensitization, U-87 MG tumor xenografts measuring 8 to 10 mm in diameter were infused in vivo with AdLacZ or Ad-EGFRCD533 as described in “Material and Methods”. This technique routinely yielded transduction efficiencies of 59 to 65% (data not shown), as determined by x-gal staining of single cells, derived from tumor digests 3 days after AdLacZ infusion. Irradiation was performed three days after Ad infusion as described. In this study, 3 fractions of 3 Gy were used based on the in vitro studies showing enhanced radiosensitization with Ad-EGFR-CD533 transduction after repeated radiation exposures (FIG. 31). Twenty-four h post-irradiation, tumors were digested to single cell suspension and ex vivo clonogenic survival was the treatment end point. The results presented in FIG. 33 show that the treatment with Ad-EGFR-CD533 and radiation resulted in a 44% survival reduction relative to the control treatment with AdLacZ and radiation (10.4 vs. 18.5% survival; P<0.001). The plating efficiencies of tumor cells from AdLacZ- and Ad-CMVEGFR-CD533-infused tumors were similar (6.79 vs. 6.14%, p>0.5).

These data demonstrate that the expression of EGFR-CD533 provides significant radiosensitization of malignant glioma cells in vitro and in vivo.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Auer K, Contessa J, Brenz-Verca S, Pirola L, Rusconi S,Cooper G, Abo A, Wymann M, Davis R J, Birrer M and Dent P. (1998). Mol. Biol. Cell, 9, 561-573.
Balaban, N., Moni, J., Shannon, M., Dang, L., Murphy, E., Goldkorn, T. Biochimica et Biophysica Acta. 1314:147-156, 1996.
Baselga J, Mendelsohn J. The epidermal growth factor receptor as a target for therapy in breast carcinoma. Breast Cancer Res. Treat., 29:127-138, 1994.
Boulton T G, Yancopoulos G D, Gregory J S, Slaughter C, Moomaw C, Hsu J and Cobb M H. (1990). Science, 249, 64-67.
Brown P H, Alani R, Preis L H, Szabo E and Birrer M J. (1993). Oncogene, 8, 877-886.
Brown et al., 1993, Oncogene, 8, 877-886
Brown J M and Lemmon M J. 1990 Cancer Res. 50, 7745-7749.
Brust D, Feden J, Farnsworth J, Amir C, Broaddus W C, Valerie K. Radiosensitization of rat glioma with brornodeoxycytidine and Adenovirus expressing HSV-TK delivered by slow positive pressure infusion. Cancer Gene Ther., 7(5), 2000.
Carmichael J, DeGraff W G, Gazdar A F, Minna J D and Mitchell J B. (1987). Cancer Res., 47, 943-946.
Carter, S., Auer, K. L., Birrer, M., Fisher, P. B., Schmidt-Ullrich, R., Valerie, K., Mikkelsen, R., and Dent, P. Inhibition of the mitogen activated protein (MAP) kinase cascade potentiates cell killing by low dose ionizing radiation in A431 human squamous carcinoma cells, Oncogene 16: 2787-2796, 1998.
Chelliah J, Freemerman A J, Wu-Pong S, Jarvis W D and Grant S. (1997). Biochem. Pharmacol., 54, 563-573.
Chen J, Willingham T, Shuford M, Bruce D, Rushing E, Smith, Y, Nisen P D. Effects of ectopic overexpression of p21 (WATI/CIPI) on aneuploidy and the malignant phenotype of human brain tumor cells. Oncogene, 13: 1395-1403, 1996.
Contessa J N, Reardon D B, Todd D, Dent P. Mikkelsen R B, Valerie K, Bowers G D and Schmidt-Ullrich R K (1999). Clin. Cancer. Res., 5, 40 5-411. (Example 1)
Denizot F and Lang R. (1986). J. Iininunol. Methods, 89, 271-277.
Dent et. al., 1999, Mol. Biol. of the Cell 10, 2493-506.
Dent P, Lavoinne A, Nakielny S, Caudwell F B, Watt P, Cohen P. 1990 Nature 348, 302-308.
Derijard B, Hibi M, Wu I H, Barrett T, Su B. Deng T, Karin M and Davis R J. (1994). Cell, 76, 1025-1037.
Fertil, B., Dertinger, H., Courdi, A. and Malaise. E. P. Radiation Res. 99: 73-84, 1984.
Gibbs et al., 2000, Science, 287, 1969-73.
Gorczyca W, Gong J and Darzynkiewicz Z. (1993). Cancer Res., 53, 1945-1951.
Gossen, M., Freundlieb, S., Bender, G., Mueller, G., Hillen, W., and Bujard, H. 1995 Science 268, 1766-1769.
Hahn G M and Little J B. 1972 Curr. Top. Radiat. Res. 8, 39-83.
Hall E J. Cell survival curves. In: Hall E J, Ryan J D, Cox K, Papadopoulos D (eds.), Radiobiology for the Radiologist, 4th ed., p. 41. Pennsylvania: J. B. Lipp micott Company, Philadelphia, 1994.
Ho K C, Lin P S. Safranin 0 counterstaining enhances the counting of P-galactosidase-expressing cells. BioTechniques, 23: 642, 1997.
Janulis M, Silberman S, Ambe-aokar A, Gutkind J S and Schultz R M. (1999). J. Biol. Chem., 274, 801-813.
Kashles, O. Yarden, Y., Fischer, R., Ullrich, A. and Schlessinger, J. A dominant negative mutation suppresses the function of normal epidermal growth factor receptors by heterodimerization. Mol. Cell Biol. 11: 1454-1463, 1991.
Kavanagh B. D., Lin P-S., Chen P. and Schmidt-Ullrich R. Radiation-induced enhanced proliferation of human squamous cancer cells in vitro: A release from inhibition by epidermal growth factor. Clin. Cancer Res. 1: 1557-1562, 1995.
Kashles O, Yarden Y, Fisher R, Ullrich A and Schlessinger J. 1991. Mol. Cell. BioL 11, 1454-63.
Kavanagh B D, Dent P, Chen P, Schmidt-Ullrich R K and Mikkelsen R B. (1998). Radiation Res., 149, 579-587.
Kavanagh, B., Dent, P., D., Chen, P., Schmidt-Ullrich, R. and Mikkelsen, R. B. Calcium-dependent stimulation of mitogen-activated protein kinase activity in A431 cells by low doses of ionizing radiation. Radiation Research 149: 579-587, 1998.
Kavanagh B D, Lin P-S, Chen P and Schmidt-Ullrich R K. (1995). Clin. Cancer Res., 1, 1557-1562.
Kawasaki H, Moriguchi T, Matsuda S, Li H Z, Nakamura S, Shimohama S, Kimura J, Gotoh Y and Nishida E. (1996). Eur. J. Biochem., 241, 315-321.
Kirshenbaum et al., 1993, J. Clin. Invest., 92,381-387.
Kyriakis J M, Banerjee P, Nikolakaki E, Dai T, Rubie E A, Ahmad M F, Avruch J and Woodgett J R. (1994). Nature, 369, 156-160.
(a) Lammering G, Reardon D, Hewit T H, Contessa J N, Hawkins W T, Lin P-S, Valerie K, Dent P, Mikkelsen R, and Schmidt-Ullrich R K. 2000, manuscript submitted.
(b) Lammering G, Valerie K, Lin P-S, Mikkelsen R, Contessa J N, Feden J P, Farnsworth J, Dent P, and Schmidt-Ullrich R K. 2000, manuscript submitted.
Lupu, R. and Lippman, M. E. The role of erbB2 signal transduction pathways in human breast cancer. Breast Cancer Res. and Treat. 27: 83-93, 1993.
Manne V, Yan N, Carboni J M. Tuornari A V, Ricca C S, Brown J G, Andahazy M L, Schmidt R J, Patel D and Zahler R. (1995). Oncogene, 10, 1763-1779.
Prabhu S S, Broaddus W C, Gillies G T, Loudon W G, Chen Z J and Smith B. 1998 Surg. Neurol. 50, 367-75.
Rausch O. and Marshall C J. (1997). Mol. Cell. Biol. 17, 1170-1179.
Reardon D R, Contessa J N, Mikkelsen R B, Valerie K, Amir C, Dent P and Schmidt-Ullrich R K. 1999. Oncogene 18, 4756-66.
Redemann, N., Holzmann, B., Rueden, T. V., Wagner, E. F., Schiessinger, J., and Ullrich, A. Anti-oncogenic activity of signaling-defective epidermal growth factor receptor mutants. Mol. Cell Biol. 12: 491-498, 1992.
Russell S J, Ye Y W, Waber P G, Shuford M, Schold S J, Nisen P D. p53 mutations, 06-alkylguanine DNA alkyltransferase activity to procarbazine in human brain tumors. Cancer, 75: 1339-1342, 1995.
Sambrook, J., Fritsch, E. F. and Maniatis, T. Molecular Cloning. Cold Spring Harbor Laboratory Press, Cold Spring Harbor, N.Y., 1989.
Schmidt-Ullrich R K, Contessa J N, Dent P, Mikkelsen R B, Valerie K, Reardon D B, Bouwers G, Lin P S. Molecular mechanisms of radiation-induced accelerated repopulation. Radiat. Oncol. Investig., 7: 321-330, 1999.
Schmidt-Ullrich, R. K., Mikkelsen, R. B., Dent, P., Todd, D. G., Valerie, K., Kavanagh, B. D., Contessa, J. N., Rorrer, W. K., and Chen, P. B. Radiation-induced proliferation of human A431 squamous carcinoma cells is dependent on EGFR tyrosine phosphorylation. Oncogene 15: 1191-1197, 1997.
Schmidt-Ullrich R. K., Valerie, K., Chan, W. and McWilliams, D. Altered expression of epidermal growth factor receptor and estrogen receptor in MCF-7 cells after single and repeated radiation exposures. Int. J. Radiat. Oncol. Biol. Phys. 29: 813-819, 1994.
Schmidt-Ullrich R. K., Valerie, K., Fogleman, B. and Walters, J. The radiation-induced autophosphorylation of epidermal growth factor receptor in human malignant mammary and squamous epithelial cells. Radiat. Res. 145: 79-83, 1996
Suy S, Anderson W B, Dent P, Chang E and Kasid U. (1997) Oncogene, 15, 53-61.
Tang P, Steck P A, Yung W K A. J. Neurooncol., 35: 303-314, 1997.
Terada K, Kaziro Y and Satoh T. (1997). J. Biol. Chem., 272, 4544-4548.
Valerie K. Viral Vectors for Gene Delivery, In: Wu-Pong, S, Rojanasakul, Y, (eds.), Biopharmaceutical Drug Design and Development, pp. 69-142. Humana Press, Inc., 1999.
Valerie K I, Brust D, Farnsworth J, Amir C, Taher M, Hershey C, Feden J. Improved radiosensitization of rat glioma cells with Adenovirus-expressed mutant HSV-TK in combination with acyclovir. Cancer Gene Ther., 7(6), 2000.
Verheij M, Bose R, Lin X H, Yao B, Jarvis W D, Grant S, Birrer M J, Szabo E, Zon L L Kyriakis J M, HaimovitzFriedman A, Fuks Z and Kolesnick R N. (1996) Nature, 380, 75-79.
Wu J, Dent P, Jelinek T, Wolfman A, Weber M J and Sturgill T W. (1993) Science, 262, 1065-1069.

1. A method for suppressing accelerated repopulation of cancer cells during radiation therapy, comprising the step of
delivering to cancer cells an effective dose of an expressible nucleic acid molecule encoding a mutant epidermal growth factor receptor.

2. The method of claim 1 wherein said mutant epidermal growth factor receptor is EGFR-CD533.

3. The method of claim 1 wherein said expressible nucleic acid molecule is a DNA molecule.

4. The method of claim 1 wherein said expressible nucleic acid molecule is in an expression cassette.

5. The method of claim 4 wherein said expression cassette is Ad-EGFR-CD533.

6. The method of claim 1 wherein said expressible nucleic acid molecule is an RNA molecule.

7. The method of claim 1 wherein said step of delivering is accomplished by administration to a patient in need thereof.

8. The method of claim 7 wherein said administration is oral.

9. The method of claim 7 wherein said administration is systemic.

10. The method of claim 7 wherein said administration is in situ at the cancer locus.

11. The method of claim 7 wherein said administration is carried out via a method selected from the group consisting of administering a viral vector, administering liposomes, and direct injection of nucleic acid.

12. The method of claim 1 wherein said cancer cells are mammary cancer cells.

13. The method of claim 1 wherein said cancer cells are glioma cells.

14. The method of claim 1 wherein said cancer cells express epidermal growth factor receptor.

15-32. (canceled)
